SEQUENCE LISTING (80 sequences)

SEQ ID NO: 1 - protein, 9 aa, unidentified
LAGLIDADG

SEQ ID NO: 2 - protein, 7 aa, synthetic construct
PKKKRKV

SEQ ID NO: 3 - protein, 7 aa, synthetic construct
PKKKRRV

SEQ ID NO: 4 - protein, 16 aa, synthetic construct
KRPAATKKAGQAKKKK

SEQ ID NO: 5 - protein, 11 aa, synthetic construct
YGRKKRRQRRR

SEQ ID NO: 6 - protein, 9 aa, synthetic construct
RKKRRQRRR

SEQ ID NO: 7 - protein, 9 aa, synthetic construct
PAAKRVKLD

SEQ ID NO: 8 - protein, 11 aa, synthetic construct
RQRRNELKRSP

SEQ ID NO: 9 - protein, 8 aa, synthetic construct
VSRKRPRP

SEQ ID NO: 10 - protein, 8 aa, synthetic construct
PPKKARED

SEQ ID NO: 11 - protein, 8 aa, synthetic construct
PQPKKKPL

SEQ ID NO: 12 - protein, 12 aa, synthetic construct
SALIKKKKKMAP

SEQ ID NO: 13 - protein, 7 aa, synthetic construct
PKQKKRK

SEQ ID NO: 14 - protein, 10 aa, synthetic construct
RKLKKKIKKL

SEQ ID NO: 15 - protein, 10 aa, synthetic construct
REKKKFLKRR

SEQ ID NO: 16 - protein, 20 aa, synthetic construct
KRKGDEVDGVDEVAKKKSKK

SEQ ID NO: 17 - protein, 17 aa, synthetic construct
RKCLQAGMNLEARKTKK

SEQ ID NO: 18 - protein, 38 aa, synthetic construct
NQSSNFGPMKGGNFGGRSSGPYGGGGQYFAKPRNQGGY

SEQ ID NO: 19 - protein, 42 aa, synthetic construct
RMRIZFKNKGKDTAELRRRRVEVSVELRKAKKDEQILKRRNV

SEQ ID NO: 20 - protein, 20 aa, synthetic construct
GRKKRRQRRRPPQPKKKRKV

SEQ ID NO: 21 - protein, 19 aa, synthetic construct
PLSSIFSRIGDPPKKKRKV

SEQ ID NO: 22 - protein, 24 aa, synthetic construct
GALFLGWLGAAGSTMGAPKKKRKV

SEQ ID NO: 23 - protein, 27 aa, synthetic construct
GALFLGFLGAAGSTMGAWSQPKKKRKV

SEQ ID NO: 24 - protein, 21 aa, synthetic construct
KETWWETWWTEWSQPKKKRKV

SEQ ID NO: 25 - protein, 11 aa, synthetic construct
YARAAARQARA

SEQ ID NO: 26 - protein, 11 aa, synthetic construct
THRLPRRRRRR

SEQ ID NO: 27 - protein, 11 aa, synthetic construct
GGRRARRRRRR

SEQ ID NO: 28 - protein, 12 aa, synthetic construct
RRQRRTSKLMKR

SEQ ID NO: 29 - protein, 27 aa, synthetic construct
GWTLNSAGYLLGKINLKALAALAKKIL

SEQ ID NO: 30 - protein, 33 aa, synthetic construct
KALAWEAKLAKALAKALAKHLAKALAKALKCEA

SEQ ID NO: 31 - protein, 16 aa, synthetic construct
RQIKIWFQNRRMKWKK

SEQ ID NO: 32 - protein, 6 aa, synthetic construct
HHHHHH

SEQ ID NO: 33 - RNA, 20 nt, synthetic construct
  source 1..20: mol_type = other RNA
gagggggaacagttctgaaa

SEQ ID NO: 34 - DNA, 23 bp, synthetic construct
  source 1..23: mol_type = other DNA
gagggggaacagttctgaaaggg

SEQ ID NO: 35 - DNA, 59 bp, synthetic construct
  source 1..59: mol_type = other DNA
  misc_difference 34: note = a, c, t, g, unknown, or other
  misc_difference 35: note = a, c, t, g, unknown, or other
  misc_difference 36: note = a, c, t, g, unknown, or other
  misc_difference 37: note = a, c, t, g, unknown, or other
  misc_difference 38: note = a, c, t, g, unknown, or other
  misc_difference 39: note = a, c, t, g, unknown, or other
tcgtcggcagcgtcagatgtgtataagagacagnnnnnnctgcttgggctgctcaatga

SEQ ID NO: 36 - DNA, 60 bp, synthetic construct
  source 1..60: mol_type = other DNA
  misc_difference 35: note = a, c, t, g, unknown, or other
  misc_difference 36: note = a, c, t, g, unknown, or other
  misc_difference 37: note = a, c, t, g, unknown, or other
  misc_difference 38: note = a, c, t, g, unknown, or other
  misc_difference 39: note = a, c, t, g, unknown, or other
  misc_difference 40: note = a, c, t, g, unknown, or other
gtctcgtgggctcggagatgtgtataagagacagnnnnnngttcctttcctccacccctt

SEQ ID NO: 37 - DNA, 23 bp, synthetic construct
  source 1..23: mol_type = other DNA
ctttaatgcgctgacttgtgagg

SEQ ID NO: 38 - DNA, 23 bp, synthetic construct
  source 1..23: mol_type = other DNA
tgacatcaattattatacatcgg

SEQ ID NO: 39 - DNA, 23 bp, synthetic construct
  source 1..23: mol_type = other DNA
agctggtcccagtaccgggaagg

SEQ ID NO: 40 - DNA, 23 bp, synthetic construct
  source 1..23: mol_type = other DNA
ggcactgcggctggaggtggggg

SEQ ID NO: 41 - DNA, 23 bp, synthetic construct
  source 1..23: mol_type = other DNA
agccgtgagtggagggagcgtgg

SEQ ID NO: 42 - DNA, 23 bp, synthetic construct
  source 1..23: mol_type = other DNA
gggtggggggagtttgctcctgg

SEQ ID NO: 43 - RNA, 20 nt, synthetic construct
  source 1..20: mol_type = other RNA
ctttaatgcgctgacttgtg

SEQ ID NO: 44 - RNA, 20 nt, synthetic construct
  source 1..20: mol_type = other RNA
tgacatcaattattatacat

SEQ ID NO: 45 - RNA, 20 nt, synthetic construct
  source 1..20: mol_type = other RNA
agctggtcccagtaccggga

SEQ ID NO: 46 - RNA, 20 nt, synthetic construct
  source 1..20: mol_type = other RNA
ggcactgcggctggaggtgg

SEQ ID NO: 47 - RNA, 20 nt, synthetic construct
  source 1..20: mol_type = other RNA
agccgtgagtggagggagcg

SEQ ID NO: 48 - RNA, 20 nt, synthetic construct
  source 1..20: mol_type = other RNA
gggtggggggagtttgctcc

SEQ ID NO: 49 - DNA, 59 bp, synthetic construct
  source 1..59: mol_type = other DNA
  misc_difference 34: note = a, c, t, g, unknown, or other
  misc_difference 35: note = a, c, t, g, unknown, or other
  misc_difference 36: note = a, c, t, g, unknown, or other
  misc_difference 37: note = a, c, t, g, unknown, or other
  misc_difference 38: note = a, c, t, g, unknown, or other
  misc_difference 39: note = a, c, t, g, unknown, or other
tcgtcggcagcgtcagatgtgtataagagacagnnnnnnctgactgctgacccctccta

SEQ ID NO: 50 - DNA, 64 bp, synthetic construct
  source 1..64: mol_type = other DNA
  misc_difference 35: note = a, c, t, g, unknown, or other
  misc_difference 36: note = a, c, t, g, unknown, or other
  misc_difference 37: note = a, c, t, g, unknown, or other
  misc_difference 38: note = a, c, t, g, unknown, or other
  misc_difference 39: note = a, c, t, g, unknown, or other
  misc_difference 40: note = a, c, t, g, unknown, or other
gtctcgtgggctcggagatgtgtataagagacagnnnnnngccacagggtagttagactctaag

SEQ ID NO: 51 - DNA, 69 bp, synthetic construct
  source 1..69: mol_type = other DNA
  misc_difference 34: note = a, c, t, g, unknown, or other
  misc_difference 35: note = a, c, t, g, unknown, or other
  misc_difference 36: note = a, c, t, g, unknown, or other
  misc_difference 37: note = a, c, t, g, unknown, or other
  misc_difference 38: note = a, c, t, g, unknown, or other
  misc_difference 39: note = a, c, t, g, unknown, or other
tcgtcggcagcgtcagatgtgtataagagacagnnnnnncatggagggcaactaaatacattctaggac

SEQ ID NO: 52 - DNA, 61 bp, synthetic construct
  source 1..61: mol_type = other DNA
  misc_difference 35: note = a, c, t, g, unknown, or other
  misc_difference 36: note = a, c, t, g, unknown, or other
  misc_difference 37: note = a, c, t, g, unknown, or other
  misc_difference 38: note = a, c, t, g, unknown, or other
  misc_difference 39: note = a, c, t, g, unknown, or other
  misc_difference 40: note = a, c, t, g, unknown, or other
gtctcgtgggctcggagatgtgtataagagacagnnnnnntcagagatggccaggttgagc

SEQ ID NO: 53 - DNA, 63 bp, synthetic construct
  source 1..63: mol_type = other DNA
  misc_difference 34: note = a, c, t, g, unknown, or other
  misc_difference 35: note = a, c, t, g, unknown, or other
  misc_difference 36: note = a, c, t, g, unknown, or other
  misc_difference 37: note = a, c, t, g, unknown, or other
  misc_difference 38: note = a, c, t, g, unknown, or other
  misc_difference 39: note = a, c, t, g, unknown, or other
tcgtcggcagcgtcagatgtgtataagagacagnnnnnngttcttgagtgtgttgagcatgcc

SEQ ID NO: 54 - DNA, 61 bp, synthetic construct
  source 1..61: mol_type = other DNA
  misc_difference 35: note = a, c, t, g, unknown, or other
  misc_difference 36: note = a, c, t, g, unknown, or other
  misc_difference 37: note = a, c, t, g, unknown, or other
  misc_difference 38: note = a, c, t, g, unknown, or other
  misc_difference 39: note = a, c, t, g, unknown, or other
  misc_difference 40: note = a, c, t, g, unknown, or other
gtctcgtgggctcggagatgtgtataagagacagnnnnnntgctcacaatctgcaccccac

SEQ ID NO: 55 - DNA, 59 bp, synthetic construct
  source 1..59: mol_type = other DNA
  misc_difference 34: note = a, c, t, g, unknown, or other
  misc_difference 35: note = a, c, t, g, unknown, or other
  misc_difference 36: note = a, c, t, g, unknown, or other
  misc_difference 37: note = a, c, t, g, unknown, or other
  misc_difference 38: note = a, c, t, g, unknown, or other
  misc_difference 39: note = a, c, t, g, unknown, or other
tcgtcggcagcgtcagatgtgtataagagacagnnnnnnggaacccaggtagccagaga

SEQ ID NO: 56 - DNA, 57 bp, synthetic construct
  source 1..57: mol_type = other DNA
  misc_difference 35: note = a, c, t, g, unknown, or other
  misc_difference 36: note = a, c, t, g, unknown, or other
  misc_difference 37: note = a, c, t, g, unknown, or other
  misc_difference 38: note = a, c, t, g, unknown, or other
  misc_difference 39: note = a, c, t, g, unknown, or other
  misc_difference 40: note = a, c, t, g, unknown, or other
gtctcgtgggctcggagatgtgtataagagacagnnnnnnggggtggggtcagacgt

SEQ ID NO: 57 - DNA, 61 bp, synthetic construct
  source 1..61: mol_type = other DNA
  misc_difference 34: note = a, c, t, g, unknown, or other
  misc_difference 35: note = a, c, t, g, unknown, or other
  misc_difference 36: note = a, c, t, g, unknown, or other
  misc_difference 37: note = a, c, t, g, unknown, or other
  misc_difference 38: note = a, c, t, g, unknown, or other
  misc_difference 39: note = a, c, t, g, unknown, or other
tcgtcggcagcgtcagatgtgtataagagacagnnnnnntggtctcccctttccagcattc

SEQ ID NO: 58 - DNA, 60 bp, synthetic construct
  source 1..60: mol_type = other DNA
  misc_difference 35: note = a, c, t, g, unknown, or other
  misc_difference 36: note = a, c, t, g, unknown, or other
  misc_difference 37: note = a, c, t, g, unknown, or other
  misc_difference 38: note = a, c, t, g, unknown, or other
  misc_difference 39: note = a, c, t, g, unknown, or other
  misc_difference 40: note = a, c, t, g, unknown, or other
gtctcgtgggctcggagatgtgtataagagacagnnnnnnaactgggcgagtgccagtct

SEQ ID NO: 59 - DNA, 60 bp, synthetic construct
  source 1..60: mol_type = other DNA
  misc_difference 34: note = a, c, t, g, unknown, or other
  misc_difference 35: note = a, c, t, g, unknown, or other
  misc_difference 36: note = a, c, t, g, unknown, or other
  misc_difference 37: note = a, c, t, g, unknown, or other
  misc_difference 38: note = a, c, t, g, unknown, or other
  misc_difference 39: note = a, c, t, g, unknown, or other
tcgtcggcagcgtcagatgtgtataagagacagnnnnnngacacacacgtcctcactctc

SEQ ID NO: 60 - DNA, 60 bp, synthetic construct
  source 1..60: mol_type = other DNA
  misc_difference 35: note = a, c, t, g, unknown, or other
  misc_difference 36: note = a, c, t, g, unknown, or other
  misc_difference 37: note = a, c, t, g, unknown, or other
  misc_difference 38: note = a, c, t, g, unknown, or other
  misc_difference 39: note = a, c, t, g, unknown, or other
  misc_difference 40: note = a, c, t, g, unknown, or other
gtctcgtgggctcggagatgtgtataagagacagnnnnnntgtctgcaggccagatgagg

SEQ ID NO: 61 - RNA, 20 nt, synthetic construct
  source 1..20: mol_type = other RNA
gaacacaaagcatagactgc

SEQ ID NO: 62 - RNA, 20 nt, synthetic construct
  source 1..20: mol_type = other RNA
gagtccgagcagaagaagaa

SEQ ID NO: 63 - RNA, 20 nt, synthetic construct
  source 1..20: mol_type = other RNA
gtcatcttagtcattacctg

SEQ ID NO: 64 - RNA, 20 nt, synthetic construct
  source 1..20: mol_type = other RNA
ggcccagactgagcacgtga

SEQ ID NO: 65 - RNA, 20 nt, synthetic construct
  source 1..20: mol_type = other RNA
cttgccccacagggcagtaa

SEQ ID NO: 66 - DNA, 63 bp, synthetic construct
  source 1..63: mol_type = other DNA
  misc_difference 34: note = a, c, t, g, unknown, or other
  misc_difference 35: note = a, c, t, g, unknown, or other
  misc_difference 36: note = a, c, t, g, unknown, or other
  misc_difference 37: note = a, c, t, g, unknown, or other
  misc_difference 38: note = a, c, t, g, unknown, or other
  misc_difference 39: note = a, c, t, g, unknown, or other
tcgtcggcagcgtcagatgtgtataagagacagnnnnnntgaacttcccaagtgagaagccag

SEQ ID NO: 67 - DNA, 60 bp, synthetic construct
  source 1..60: mol_type = other DNA
  misc_difference 35: note = a, c, t, g, unknown, or other
  misc_difference 36: note = a, c, t, g, unknown, or other
  misc_difference 37: note = a, c, t, g, unknown, or other
  misc_difference 38: note = a, c, t, g, unknown, or other
  misc_difference 39: note = a, c, t, g, unknown, or other
  misc_difference 40: note = a, c, t, g, unknown, or other
gtctcgtgggctcggagatgtgtataagagacagnnnnnntggcaggacgtctgcccaat

SEQ ID NO: 68 - DNA, 55 bp, synthetic construct
  source 1..55: mol_type = other DNA
  misc_difference 34: note = a, c, t, g, unknown, or other
  misc_difference 35: note = a, c, t, g, unknown, or other
  misc_difference 36: note = a, c, t, g, unknown, or other
  misc_difference 37: note = a, c, t, g, unknown, or other
  misc_difference 38: note = a, c, t, g, unknown, or other
  misc_difference 39: note = a, c, t, g, unknown, or other
tcgtcggcagcgtcagatgtgtataagagacagnnnnnnccccagtggctgctct

SEQ ID NO: 69 - DNA, 57 bp, synthetic construct
  source 1..57: mol_type = other DNA
  misc_difference 35: note = a, c, t, g, unknown, or other
  misc_difference 36: note = a, c, t, g, unknown, or other
  misc_difference 37: note = a, c, t, g, unknown, or other
  misc_difference 38: note = a, c, t, g, unknown, or other
  misc_difference 39: note = a, c, t, g, unknown, or other
  misc_difference 40: note = a, c, t, g, unknown, or other
gtctcgtgggctcggagatgtgtataagagacagnnnnnnccaggcctccccaaagc

SEQ ID NO: 70 - DNA, 69 bp, synthetic construct
  source 1..69: mol_type = other DNA
  misc_difference 34: note = a, c, t, g, unknown, or other
  misc_difference 35: note = a, c, t, g, unknown, or other
  misc_difference 36: note = a, c, t, g, unknown, or other
  misc_difference 37: note = a, c, t, g, unknown, or other
  misc_difference 38: note = a, c, t, g, unknown, or other
  misc_difference 39: note = a, c, t, g, unknown, or other
tcgtcggcagcgtcagatgtgtataagagacagnnnnnntgttagccaacatacagaagtcaggaatgc

SEQ ID NO: 71 - DNA, 64 bp, synthetic construct
  source 1..64: mol_type = other DNA
  misc_difference 35: note = a, c, t, g, unknown, or other
  misc_difference 36: note = a, c, t, g, unknown, or other
  misc_difference 37: note = a, c, t, g, unknown, or other
  misc_difference 38: note = a, c, t, g, unknown, or other
  misc_difference 39: note = a, c, t, g, unknown, or other
  misc_difference 40: note = a, c, t, g, unknown, or other
gtctcgtgggctcggagatgtgtataagagacagnnnnnnttcagaccatagcacttcccttcc

SEQ ID NO: 72 - DNA, 59 bp, synthetic construct
  source 1..59: mol_type = other DNA
  misc_difference 34: note = a, c, t, g, unknown, or other
  misc_difference 35: note = a, c, t, g, unknown, or other
  misc_difference 36: note = a, c, t, g, unknown, or other
  misc_difference 37: note = a, c, t, g, unknown, or other
  misc_difference 38: note = a, c, t, g, unknown, or other
  misc_difference 39: note = a, c, t, g, unknown, or other
tcgtcggcagcgtcagatgtgtataagagacagnnnnnnacctccctaggtgctggctt

SEQ ID NO: 73 - DNA, 60 bp, synthetic construct
  source 1..60: mol_type = other DNA
  misc_difference 35: note = a, c, t, g, unknown, or other
  misc_difference 36: note = a, c, t, g, unknown, or other
  misc_difference 37: note = a, c, t, g, unknown, or other
  misc_difference 38: note = a, c, t, g, unknown, or other
  misc_difference 39: note = a, c, t, g, unknown, or other
  misc_difference 40: note = a, c, t, g, unknown, or other
gtctcgtgggctcggagatgtgtataagagacagnnnnnngctgatgtgggctgcctaga

SEQ ID NO: 74 - DNA, 59 bp, synthetic construct
  source 1..59: mol_type = other DNA
  misc_difference 34: note = a, c, t, g, unknown, or other
  misc_difference 35: note = a, c, t, g, unknown, or other
  misc_difference 36: note = a, c, t, g, unknown, or other
  misc_difference 37: note = a, c, t, g, unknown, or other
  misc_difference 38: note = a, c, t, g, unknown, or other
  misc_difference 39: note = a, c, t, g, unknown, or other
tcgtcggcagcgtcagatgtgtataagagacagnnnnnngccaatctactcccaggagc

SEQ ID NO: 75 - DNA, 62 bp, synthetic construct
  source 1..62: mol_type = other DNA
  misc_difference 35: note = a, c, t, g, unknown, or other
  misc_difference 36: note = a, c, t, g, unknown, or other
  misc_difference 37: note = a, c, t, g, unknown, or other
  misc_difference 38: note = a, c, t, g, unknown, or other
  misc_difference 39: note = a, c, t, g, unknown, or other
  misc_difference 40: note = a, c, t, g, unknown, or other
gtctcgtgggctcggagatgtgtataagagacagnnnnnnaggcagagagagtcagtgccta

SEQ ID NO: 76 - DNA, 23 bp, synthetic construct
  source 1..23: mol_type = other DNA
gtcatcttagtcattacctgagg

SEQ ID NO: 77 - DNA, 23 bp, synthetic construct
  source 1..23: mol_type = other DNA
gaacacaaagcatagactgcggg

SEQ ID NO: 78 - DNA, 23 bp, synthetic construct
  source 1..23: mol_type = other DNA
ggcccagactgagcacgtgatgg

SEQ ID NO: 79 - DNA, 22 bp, synthetic construct
  source 1..22: mol_type = other DNA
gagtccgagcagaagaagaagg

SEQ ID NO: 80 - DNA, 23 bp, synthetic construct
  source 1..23: mol_type = other DNA
cttgccccacagggcagtaacgg

(19) United States
(12) Patent Application Publication
(10) Pub. No.: US 2025/0066816 A1
(43) Pub. Date: Feb. 27, 2025

(54) ELECTROPORATION ENHANCERS FOR CRISPR-CAS SYSTEMS

(71) Applicant: SIGMA-ALDRICH CO. LLC, St. Louis, MO (US)

(72) Inventors: Fuqiang Chen, St. Louis, MO (US); Graeme Garvey, Defiance, MO (US); Xiao Chen, St. Louis, MO (US)

(21) Appl. No.: 18/704,247

(22) PCT Filed: Nov. 1, 2022
(86) PCT No.: PCT/US2022/079037

§ 371 Date: Jun. 12, 2024

Related U.S. Application Data
(60) Provisional application No. 63/274,272, filed on Nov. 1, 2021.

(51) Int. Cl. C12N 15/87; C12N 9/22; C12N 15/11

(52) U.S. Cl. CPC C12N 15/87; C12N 9/22; C12N 15/111; C07K 2319/09; C12N 2310/20

(57) ABSTRACT

This disclosure provides methods and compositions for improving the genome modification efficiency of programmable DNA modification proteins during transfection (e.g., electroporation).

### CROSS-REFERENCE TO RELATED APPLICATIONS

The present application claims the benefit of priority of U.S. Provisional Patent Application No. 63/274,272 filed Nov. 1, 2021, the entirety of which is incorporated herein by reference.

### FIELD

The present disclosure generally relates to methods and compositions for increasing the efficiency of targeted genome modification, targeted transcriptional regulation, and/or targeted epigenetic modification.

### BACKGROUND

Programmable endonucleases have become an important tool for targeted genome engineering or modification in eukaryotes. RNA-guided clustered regularly interspaced short palindromic repeats (CRISPR) systems have emerged as a new generation of genome modification tools. These programmable endonucleases provide unprecedented simplicity and versatility as compared to previous generations of nucleases such as zinc finger nucleases (ZFNs) and transcription activator-like effector nucleases (TALENs).

CRISPR-Cas systems have been widely adopted as an endonuclease for genome modification in diverse cell types and organisms. More recently, the use of CRISPR-Cas systems has been further expanded to base editing and prime editing by fusion of Cas polypeptides with other functional domains, such as cytidine deaminase, adenosine deaminase, and reverse transcriptase. While plasmid DNA has traditionally been used as a vehicle for delivering these genome modification agents into the cell, ribonucleoprotein (RNP) complexes consisting of recombinant Cas proteins and single guide RNA (sgRNA) have recently become an important delivery format in many gene editing applications. RNP complex delivery can minimize or even eliminate the risk of exogenous DNA integration into the host genome, reduce off-target effects, and alleviate transfection associated cytotoxicity in some cell types, such as T-cells, which are highly sensitive to plasmid DNA.

One limitation in using CRISPR-Cas (e.g., Cas9, Cas12) RNP complexes for genome modification can be higher variable editing efficiency as compared with plasmid DNA delivery. Several factors may contribute to this drawback. Particularly, the retention of RNP complexes on the extracellular matrix (ECM) can reduce the delivery efficiency, and the aggregation of RNP complexes in the cytoplasm can substantially limit the amount of the complexes for transportation into the nucleus for gene editing. Integrated DNA Technologies, Inc. (IDT) has developed a 100-nt synthetic single-stranded DNA oligo as an electroporation enhancer for CRISPR-Cas9 and CRISPR-Cas12 RNP complex delivery to improve editing efficiency. However, these DNA-based enhancers pose the risk of exogenous DNA integration into the host genome and may interfere with gene editing assays.

### SUMMARY OF THE DISCLOSURE

Among the various aspects of the present disclosure is the provision of methods and compositions for improving the genome modification efficiency of programmable DNA modification proteins during transfection (e.g., electroporation).

Briefly, therefore, the present disclosure is directed to methods for enhancing genome editing efficiency. The methods involve introducing, by electroporation, a programmable DNA modification system to a eukaryotic cell in the presence of a polyanionic polymer or salt thereof. In certain embodiments, the programmable DNA modification system is an RNP complex. In certain embodiments, the electroporation enhancer is RNA- and DNA-free; that is little or no detectable nucleic acid is present in the enhancer composition or solution. In these and other embodiments, the polyanionic polymer or salt thereof is dextran sulfate sodium salt.

Another aspect of the disclosure is directed to eukaryotic cells, prepared in accordance with the methods described herein.

Yet another aspect of the disclosure is directed to a transfection composition comprising a programmable DNA modification system, a eukaryotic cell, and a polyanionic polymer or salt thereof (e.g., dextran sulfate and salts thereof). Another aspect of the disclosure is directed to a transfection composition comprising two or more polyanionic polymers or salts thereof (e.g., dextran sulfate and salts thereof and another polyanionic polymer(s) or salt(s) thereof).

Other aspects of the disclosure include kits including electroporation solution(s) and one or more of Cas protein(s), reconstitution solution(s), protein dilution solution(s), and optionally other components, as described herein. In one particular aspect, the kit comprises a Cas protein, a solution including a polyanionic polymer or salt thereof, and one or more buffer solutions. In one embodiment, the kit comprises a recombinant Cas protein, a dextran sulfate (e.g., sodium salt) solution, a reconstitution solution, a dilution solution, and instructions for use of the same for electroporation.

Other objects and features will be in part apparent and in part pointed out hereinafter.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is a graph depicting the mutation frequencies produced by an SpCas9 recombinant protein and a synthetic sgRNA on a human RelA target site under different amounts of dextran sulfate used in nucleofection of human K562 cells.

FIG. 2 is a graph depicting the mutation frequencies generated by an SpCas9 recombinant protein and six synthetic sgRNAs on human CAR19, CCR5, CHI3L1, HEKSite4, POR23, and VEGFA target sites in the presence and absence of dextran sulfate during nucleofection of human K562 cells.

FIGS. 3A-3F are graphs depicting the C to T base transition editing frequencies generated by a recombinant cytosine base editor fusion protein, comprising an SpCas9 D10A nickase and a human APOBEC3A cytidine deaminase, and six synthetic sgRNAs on human CCR5 (FIG. 3A), RNF2 (FIG. 3B), HEK293_site 2 (FIG. 3C), HEK293_site 3 (FIG. 3D), EMX1 (FIG. 3D), and HBB (FIG. 3F) target sites in the presence and absence of dextran sulfate during nucleofection of human HEK293 cells. These figures disclose SEQ ID NOS 38, and 76-80, respectively, in order of appearance (see also Example 3, below).

FIG. 4A is a graph depicting the genome editing efficiency by an SpCas9 recombinant protein and a synthetic guide RNA on a HEKSite4 target side in human fibroblast cells in the presence of different amounts of pentosan polysulfate. FIG. 4B is a graph depicting the genome editing efficiency by an SpCas9 recombinant protein and a synthetic guide RNA on a HEKSite4 target site in human HEK293 cells in the presence of different amounts of pentosan polysulfate.

FIG. 5A is a graph depicting the genome editing efficiency by an SpCas9 recombinant protein and a synthetic guide RNA on a HEKSite4 target site in human fibroblast cells in the presence of different amounts of heparan sulfate. FIG. 5B is a graph depicting the genome editing efficiency by an SpCas9 recombinant protein and a synthetic guide RNA on a HEKSite4 target site in human HEK293 cells in the presence of different amounts of heparan sulfate.

FIG. 6 is a graph depicting the effects of nine different compounds on the genome editing efficiency by an SpCas9 recombinant protein and a synthetic guide RNA on a HEKSite4 target site in human HEK293 cells.

FIG. 7 is a graph depicting the effect of dextran sulfate sodium salt on the genome editing efficiency by an SpCas9 recombinant protein and a synthetic guide RNA on a PD-1 target site in human primary T cells.

### DETAILED DESCRIPTION

CRISPR-Cas systems, for example, CRISPR-Cas ribonucleoprotein (RNP) complexes assembled from recombinant Cas protein and guide RNAs, have become an important reagent in genome modification in diverse cell types and organisms. The present disclosure provides certain polyanionic polymers and salts thereof (such as sulfated polysaccharides), for use as an enhancer for improving the genome modification efficiency of programmable DNA modification proteins during transfection (e.g., electroporation). In certain embodiments, the programmable DNA modification protein is a Cas protein including nucleases, nickases, and base editors, e.g., delivered as RNP complexes. The methods disclosed herein may be also applied to the electroporation delivery of other recombinant proteins to enhance genome editing efficiency or relevant biological effects.

One aspect of the present disclosure is directed to a method for improving genome editing efficiency. In general, the method involves introducing a programmable DNA modification system to a eukaryotic cell in the presence of a polyanionic polymer or salt thereof. Typically, the programmable DNA modification system is introduced to the eukaryotic cell via electroporation. In accordance with the methods described herein, genome editing efficiency is enhanced relative to an otherwise identical method in the absence of the polyanionic polymer or salt thereof. Other aspects of the disclosure include compositions (including electroporation solutions) for enhancing genome editing efficiency.

### Polyanionic Polymers as Electroporation Enhancers

In accordance with the methods, compositions, and uses disclosed herein, polyanionic polymers are used to improve or enhance the genome modification efficiency of programmable DNA modification systems. Such programmable DNA modification systems include, for example, RNA-guided clustered regularly interspaced short palindromic repeats (CRISPR)/CRISPR-associated (Cas) (CRISPR-Cas) nuclease systems, CRISPR-Cas dual nickase systems, zinc finger nucleases (ZFNs), transcription activator-like effector nucleases (TALENs), meganucleases, fusion proteins including a programmable DNA binding domain linked to a nuclease domain, and/or fusion proteins comprising a programmable DNA binding domain linked to a non-nuclease domain. In one particular embodiment, the programmable DNA modification system is a CRISPR-Cas nuclease system. In another particular embodiment, the programmable DNA modification system is a CRISPR-Cas9 or a CRISPR-Cas12 nuclease system. In yet another particular embodiment, the programmable DNA modification is a CRISPR-Cas9 nuclease or nickase system.

The polyanionic polymers are typically added to, or otherwise present in, a conventional electroporation solution before carrying out the electroporation process. In various embodiments, the polyanionic polymer or salt thereof is added to an electroporation solution either before or after cells are resuspended in the solution and the electroporation voltage is supplied. As illustrated in the examples below, for example, using a conventional electroporation system, the polyanionic polymer or salt thereof (e.g., dextran sulfate sodium salt) is added to a standard electroporation solution, such as Nucleofector™ Solution V (Lonza), and the cells are then resuspended in the polyanionic polymer-containing electroporation solution prior to electroporation (also referred to in the art, and herein, as nucleofection). Alternatively, the cells can first be resuspended in the electroporation solution and the polyanionic polymer(s) added to the cell suspension prior to electroporation/nucleofection. Those of skill in the art will appreciate that there are many different electroporation systems suitable for delivering programmable DNA modification systems into the cell for genome modification and the present disclosure including methods, compositions, uses, and kits can be applied to these systems.

The polyanionic polymer may be one or more organic polymers or salts thereof. Examples of suitable organic polymers include, but are not limited to, polysaccharide polymers. In one embodiment, the polysaccharide polymer is selected from carrageenan (e.g., K-, I-, and/or L-carrageenans), cellulose (e.g., carboxymethylcellulose), chondroitin, collagen, dextran, fucoidan, heparan, heparin, glucosamine, laminarin, pentosan, and derivatives and/or salts thereof. In some embodiments, the polyanionic polymer is a polysaccharide sulfate or a salt thereof, wherein the repeating saccharide unit includes at least one sulfate group. Examples of sulfated polysaccharides may be selected from the group consisting of dextran sulfate, fucoidan sulfate, heparan sulfate, heparin sulfate, chondroitin sulfate, dermatan sulfate, and salts thereof. In a particular embodiment, the polyanionic polymer is the sodium salt of a sulfated polysaccharide.

In a particular embodiment, the polyanionic polymer is a polysulfate polymer such as dextran sulfate or salts thereof (e.g., dextran sulfate sodium salt). In some preferred embodiments, the polyanionic polymer is dextran sulfate or a salt thereof, such as dextran sulfate sodium salt.

Combinations of polyanionic polymers and salts thereof are contemplated in the present disclosure. For instance, the polyanionic polymers and salts thereof could be a dextran sulfate (e.g., dextran sulfate sodium salt) and another one or more polyanionic polymer or polysaccharide sulfate such as fucoidan sulfate, heparan sulfate, heparin sulfate, chondroitin sulfate, dermatan sulfate, and salts thereof.

Irrespective of the particular polyanionic polymer(s) or sulfated polysaccharide(s) chosen for use in the electroporation enhancing solution as described herein, the average molecular weight of the polyanionic polymer or a salt thereof is typically from about 1 kDa to about 1,000 kDa. In general, average molecular weight is defined as the total weight of polymer divided by the total number of molecules. The molecular weight of dextran sulfate sodium salt, for example, is typically determined by gel permeation chromatography (size exclusion chromatography using dextran as reference). In one particular embodiment, the average molecular weight of the polyanionic polymer or a salt thereof is from about 5 kDa to about 500 kDa. For example, in one embodiment, the average molecular weight of the polyanionic polymer or a salt thereof can be about 5 kDa, 15 kDa, 25 kDa, 35 kDa, 45 kDa, 55 kDa, 65 kDa, 75 kDa, 85 kDa, 95 kDa, 105 kDa, 115 kDa, 125 kDa, 135 kDa, 145 kDa, 155 kDa, 165 kDa, 175 kDa, 185 kDa, 195 kDa, 205 kDa, 215 kDa, 225 kDa, 235 kDa, 245 kDa, 255 kDa, 265 kDa, 275 kDa, 285 kDa, 295 kDa, 305 kDa, 315 kDa, 325 kDa, 335 kDa, 345 kDa, 355 kDa, 365 kDa, 375 kDa, 385 kDa, 395 kDa, 405 kDa, 415 kDa, 425 kDa, 435 kDa, 445 kDa, 455 kDa, 465 kDa, 475 kDa, 485 kDa, 495 kDa, or 500 kDa.

In another embodiment, the average molecular weight of the polyanionic polymer or a salt thereof is greater than about 5 kDa, greater than about 10 kDa, greater than about 25 kDa, greater than about 50 kDa, greater than 75 kDa, greater than about 100 kDa, greater than about 125 kDa, greater than about 150 kDa, greater than about 175 kDa, greater than about 200 kDa, greater than about 225 kDa, greater than about 250 kDa, greater than about 275 kDa, greater than about 300 kDa, greater than about 325 kDa, greater than about 350 kDa, greater than about 375 kDa, greater than about 400 kDa, greater than about 425 kDa, greater than about 450 kDa, greater than about 475 kDa, or greater than about 500 kDa.

In another embodiment, the average molecular weight of the polyanionic polymer or a salt thereof is from about 5 kDa to about 500 kDa, from about 25 kDa to about 250 kDa, from about 50 kDa to about 200 kDa, from about 75 kDa to about 175 kDa, or from about 100 kDa to about 150 KDa.

In another embodiment, the average molecular weight of the polyanionic polymer or a salt thereof is from about 5 kDa to about 25 kDa, from about 5 kDa to about 20 kDa, or from about 5 kDa to about 15 kDa.

In still other embodiments, the average molecular weight of the polyanionic polymer or a salt thereof is not narrowly critical.

The polyanionic polymers may be derived in the sodium salt forms or other salt forms and are typically dissolved in water or a buffer (e.g., a standard electroporation solution including dextran sulfate sodium salt).

The amount of polyanionic polymer or salt thereof used in each transfection may typically range from 0.1 μg to 10 μg per 100 μL of cells suspended in an electroporation solution. In one preferred embodiment, the amount may range from 0.2 μg to 1 μg per 100 μL of cells. The optimal amount can and will vary dependent on the cell type, the electroporation system, and the type of programmable DNA modification systems such as CRISPR systems (e.g., RNPs) used.

When the polyanionic material is a sulfated polysaccharide or salt thereof, each saccharide unit (i.e., repeating unit) of the sulfated polysaccharide may include 1 to 3 anionic groups. For example, when the sulfated polysaccharide is dextran sulfate, the dextran sulfate repeating unit may contain 3 sulfate groups.

It will be understood that the polyanionic polymer or salt thereof (e.g., sulfated polysaccharide or salt thereof) is to be used at concentrations and durations of use that are substantially non-toxic to cells. Similarly, it will be understood that the substantially non-toxic concentrations and durations of use may differ dependent upon the cell type, electroporation conditions, and/or other factors. In certain embodiments, the transfection or electroporation solution comprising a polyanionic polymer or salt thereof is RNA- and DNA-free; that is little or no detectable nucleic acid is present in the solution.

### Electroporation

In general, electroporation is used in both in vitro and in vivo procedures to introduce foreign material into living cells. For in vitro applications, a sample of live cells is first mixed with the agent(s) of interest and placed between electrodes such as parallel plates. Then, the electrodes apply an electrical field to the cell/implant mixture. Examples of systems that perform in vitro electroporation include Lonza Nucleofector® systems, MaxCyte Flow Electroporation® systems, and ThermoFisher Neon Transfection Systems.

The known electroporation techniques (both in vitro and in vivo) function by applying a brief high voltage pulse to electrodes positioned around the treatment region. The electric field generated between the electrodes causes the cell membranes to temporarily become porous, whereupon molecules of the agent(s) of interest enter the cells. In known electroporation applications, this electric field comprises a single square wave pulse on the order of 1000 V/cm, of about 100 us duration. Such a pulse may be generated, for example, in known applications of the Lonza Nucleofector® system (or similar device) in accordance with standard techniques known in the art or detailed in the electroporation device literature.

The application of the electric field is often in the form of multiple pulses such as double pulses of the same strength and capacitance or sequential pulses of varying strength and/or capacitance. As used herein, the term “pulse” includes one or more electric pulses at variable capacitance and voltage and including exponential and/or square wave and/or modulated wave/square wave forms.

The electric pulse is typically delivered as a waveform selected from an exponential wave form, a square wave form, a modulated wave form and a modulated square wave form.

A preferred embodiment employs direct current at low voltage. For example, an electric field may be applied at a field strength of between 1V/cm and 20V/cm, for a period of 100 milliseconds or more, preferably 15 milliseconds or more. In general, however, it will be understood by those of skill in the art that the electrical and time parameters for the electroporation are chosen in accordance with the manufacturer's recommended settings and/or installed programs for a given transfection application (e.g., cell type, protein type, etc.). In one particular embodiment, the electroporation is performed on mammalian (e.g., human) cells using an RNP complex(es).

Electroporation techniques for programmable DNA modification proteins such as CRISPR-Cas systems (e.g., Cas9, Cas12) are generally known in the art, apart from the electroporation enhancements described herein.

### Programmable DNA Modification Proteins

A programmable DNA modification protein is a protein targeted to bind a specific target sequence in chromosomal DNA, where it modifies the DNA, or a protein associated with the DNA, at or near the targeted sequence. Thus, a programmable DNA modification protein may typically comprise a programmable DNA binding domain and a catalytically active modification domain.

The DNA binding domain of the programmable DNA modification protein is programmable, meaning that it can be designed or engineered to recognize and bind different DNA sequences. In some embodiments, for example, DNA binding is mediated by interactions between the DNA modification protein and the target DNA. Thus, the DNA binding domain can be programmed to bind a DNA sequence of interest by protein engineering. In other embodiments, for example, DNA binding is mediated by a guide RNA that interacts with the DNA modification protein and the target DNA. In such instances, the programmable DNA binding protein can be targeted to a DNA sequence of interest by designing the appropriate guide RNA.

A variety of modification domains can be included in the programmable DNA modification protein. In some embodiments, the modification domain has nuclease activity and can cleave one or both strands of a double-stranded DNA sequence. The DNA break can then be repaired by a cellular DNA repair process such as non-homologous end-joining (NHEJ) or homology-directed repair (HDR), such that the DNA sequence can be modified by a deletion, insertion, and/or substitution of at least one base pair. Examples of programmable DNA modification proteins having nuclease activity include, without limit, CRISPR nucleases (or nickases), zinc finger nucleases, transcription activator-like effector nucleases, meganucleases, and a programmable DNA binding domain linked to a nuclease domain. Programmable DNA modification proteins having nuclease activity are detailed below.

In other embodiments, the modification domain of the programmable DNA modification protein has non-nuclease activity (e.g., epigenetic modification activity or transcriptional regulation activity) such that the programmable DNA modification protein modifies the structure and/or activity of the DNA and/or protein(s) associated with the DNA. Thus, the programmable DNA modification protein can comprise a programmable DNA binding domain linked to a non-nuclease domain. Such proteins are detailed below.

The programmable DNA modification proteins can comprise wild-type or naturally occurring DNA binding and/or modification domains, modified or engineered versions of naturally occurring DNA binding and/or modification domains, synthetic or artificial DNA binding and/or modification domains, and combinations thereof.

Programmable DNA Modification Proteins with Nuclease Activity

Examples of programmable DNA modification proteins having nuclease activity include, without limit, CRISPR nucleases, zinc finger nucleases, transcription activator-like effector nucleases, meganucleases, and programmable DNA binding domains linked to nuclease domains.

CRISPR Nucleases. The CRISPR nuclease(s) can be derived from a type I, type II (i.e., Cas9), type III, type V (i.e., Cas12/Cpf1), or type VI (i.e., Cas13) CRISPR protein, which are present in various bacteria and archaea. In further embodiments, the CRISPR nuclease can be derived from an archaeal CRISPR system, a CRISPR/CasX system, or a CRISPR/CasY system (Burstein et al., Nature, 2017, 542 (7640): 237-241). In various embodiments, the CRISPR nuclease (or nickase, see below) can be from Streptococcus sp. (e.g., S. pyogenes, S. thermophilus, S. pasteurianus), Campylobacter sp. (e.g., Campylobacter jejuni), Francisella sp. (e.g., Francisella novicida), Acaryochloris sp., Acetohalobium sp., Acidaminococcus sp., Acidithiobacillus sp., Alicyclobacillus sp., Allochromatium sp., Ammonifex sp., Anabaena sp., Arthrospira sp., Bacillus sp., Burkholderiales sp., Caldicelulosiruptor sp., Candidatus sp., Clostridium sp., Crocosphaera sp., Cyanothece sp., Exiguobacterium sp., Finegoldia sp., Ktedonobacter sp., Lachnospiraceae sp., Lactobacillus sp., Lyngbya sp., Marinobacter sp., Methanohalobium sp., Microscilla sp., Microcoleus sp., Microcystis sp., Natranaerobius sp., Neisseria sp., Nitrosococcus sp., Nocardiopsis sp., Nodularia sp., Nostoc sp., Oscillatoria sp., Polaromonas sp., Pelotomaculum sp., Pseudoalteromonas sp., Petrotoga sp., Prevotella sp., Staphylococcus sp., Streptomyces sp., Streptosporangium sp., Synechococcus sp., Thermosipho sp., or Verrucomicrobia sp.

Cas molecules of a variety of species can be used in the methods, compositions, uses, and kits described herein, including Cas molecules derived from S. pyogenes, S. aureus, N. meningitidis, S. thermophiles, Acidovorax avenae, Actinobacillus pleuropneumonias, Actinobacillus succinogenes, Actinobacillus suis, Actinomyces sp., Cychphilusdenitrificans, Aminomonas paucivorans, Bacillus cereus, Bacillus smithii, Bacillus thuringiensis, Bacteroides sp., Blastopirellula marina, Bradyrhizobium sp., Brevibacillus laterospoxus, Campylobacter coli, Campylobacter jejuni, Campylobacter lari, Candidatus puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, Gammaproteobacterium, Gluconacetobacter diazotrophicus, Haemophilus parainfluenzae, Haemophilus sputomm, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria meningitidis, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus aureus, Staphylococcus lugdunensis, Streptococcus sp., Subdoligranulum sp., Tistrella mobilis, Treponema sp., or Verminephrobacter eiseniae.

The CRISPR nuclease can be a wild type or naturally occurring protein. Alternatively, the CRISPR nuclease can be engineered to have improved specificity, altered PAM specificity, decreased off-target effects, increased stability, and the like.

In some embodiments, the Cas protein is a naturally occurring Cas protein. In other embodiments, the Cas protein is an engineered Cas protein. In some embodiments, the Cas endonuclease is selected from the group consisting of C2C1, C2C3, Cpf1 (also referred to herein as Cas12a), Cas12b, Cas12c, Cas12d, Cas12e, Cas13a, Cas13b, Cas13c, Cas13d, CasI, CasI B, Cas2, Cas3, Cas4, Cas5, Cash, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4.

In some embodiments, the Cas protein is an endoribonuclease such as a Cas13 protein. In some embodiments, the Cas13 protein is a Cas13a (Abudayyeh et al., Nature 550 (2017), 280-284), Cas13b (Cox et al., Science (2017) 358:6336, 1019-1027), Cas13c (Cox et al., Science (2017) 358:6336, 1019-1027), or Cas13d (Zhang et al., Cell 175 (2018), 212-223) protein.

In some embodiments, the Cas protein is a wild type or naturally occurring Cas9 protein or a Cas9 ortholog. Wild type Cas9 is a multi-domain enzyme that uses an HNH nuclease domain to cleave the target strand of DNA and a RuvC-like domain to cleave the non-target strand. Binding of WT Cas9 to DNA based on gRNA specificity results in double-stranded DNA breaks that can be repaired by non-homologous end joining (NHEJ) or homology-directed repair (HDR).

In some embodiments, the naturally occurring Cas9 polypeptide is selected from the group consisting of SpCas9, SpCas9-HF1, SpCas9-HF2, SpCas9-HF3, SpCas9-HF4, SaCas9, FnCpf, FnCas9, eSpCas9, and NmeCas9. In some embodiments, the Cas9 protein comprises an amino acid sequence having at least 60%, 65%, 70%, 75%, 80%, 85%, 90%, 95%, 96%, 97%, 98%, 99%, or 100% sequence identity to a Cas9 amino acid sequence described in Chylinski et al., RNA Biology 2013 10:5, 727-737; Hou et al., PNAS Early Edition 2013, 1-6).

In some embodiments, the Cas polypeptide comprises one or more of the following activities: (a) a nickase activity, i.e., the ability to cleave a single strand, e.g., the non-complementary strand or the complementary strand, of a nucleic acid molecule; (b) a double stranded nuclease activity, i.e., the ability to cleave both strands of a double stranded nucleic acid and create a double stranded break, which in an embodiment is the presence of two nickase activities; (c) an endonuclease activity; (d) an exonuclease activity; and/or (e) a helicase activity, i.e., the ability to unwind the helical structure of a double stranded nucleic acid. In other embodiments, the Cas protein may be dead or inactive (e.g., dCas).

In some embodiments, the Cas polypeptide is fused to heterologous polypeptide/proteins that has base deaminase activity.

In some embodiments, different Cas proteins (i.e., Cas9 proteins from various species) may be advantageous to use in the various provided methods in order to capitalize on various enzymatic characteristics of the different Cas proteins (e.g., for different PAM sequence preferences; for increased or decreased enzymatic activity; for an increased or decreased level of cellular toxicity; to change the balance between NHEJ, homology-directed repair, single strand breaks, double strand breaks, etc.).

In some embodiments, the Cas protein is a Cas9 protein derived from S. pyogenes and recognizes the PAM sequence motif NGG, NAG, and/or NGA (Mali et al, Science 2013; 339 (6121): 823-826). In some embodiments, the Cas protein is a Cas9 protein derived from S. thermophiles and recognizes the PAM sequence motif NGGNG and/or NNAGAAW (W=A or T) (See, e.g., Horvath et al, Science, 2010; 327 (5962): 167-170, and Deveau et al, J Bacteriol 2008; 190 (4): 1390-1400). In some embodiments, the Cas protein is a Cas9 protein derived from S. mutans and recognizes the PAM sequence motif NGG and/or NAAR (R=A or G) (See, e.g., Deveau et al, J BACTERIOL 2008; 190 (4): 1390-1400). In some embodiments, the Cas protein is a Cas9 protein derived from S. aureus and recognizes the PAM sequence motif NNGRR (R=A or G). In some embodiments, the Cas protein is a Cas9 protein derived from S. aureus and recognizes the PAM sequence motif N GRRT (R=A or G). In some embodiments, the Cas protein is a Cas9 protein derived from S. aureus and recognizes the PAM sequence motif N GRRV (R=A or G). In some embodiments, the Cas protein is a Cas9 protein derived from N. meningitidis and recognizes the PAM sequence motif N GATT or N GCTT (R=A or G, V=A, G or C) (See, e.g., Hou et ah, PNAS 2013, 1-6). In the aforementioned embodiments, N can be any nucleotide residue, e.g., any of A, G, C or T. In some embodiments, the Cas protein is a Cas13a protein derived from Leptotrichia shahii and recognizes the PFS sequence motif of a single 3′ A, U, or C.

In some embodiments, the Cas polypeptides described herein can be engineered to alter the PAM/PFS specificity of the Cas polypeptide. In some embodiments, a mutant Cas polypeptide has a PAM/PFS specificity that is different from the PAM/PFS specificity of the parental Cas polypeptide. For example, a naturally occurring Cas protein can be modified to alter the PAM/PFS sequence that the mutant Cas polypeptide recognizes to decrease off target sites, improve specificity, or eliminate a PAM/PFS recognition requirement. In some embodiments, a Cas protein can be modified to increase the length of the PAM/PFS recognition sequence. In some embodiments, the length of the PAM recognition sequence is at least 4, 5, 6, 7, 8, 9, 10 or 15 amino acids in length. Cas polypeptides that recognize different PAM/PFS sequences and/or have reduced off-target activity can be generated using directed evolution. Exemplary methods and systems that can be used for directed evolution of Cas polypeptides are described, e.g., in Esvelt et al. Nature 2011, 472 (7344): 499-503.

In various embodiments, the Cas protein can be a Cas mutant in which one or more mutations and/or deletions are present relative to a wild-type version of the Cas protein. For example, one or more of the following amino acid positions can be mutated (with reference to the numbering system of Streptococcus pyogenes Cas9, SpCas9: K526, K562, K652, R661, R691, R780, K810, K848, K855, K1003, and K1060. Exemplary Cas mutants are described in International PCT Publication No. WO 2015/161276; International PCT Publication No. WO 2021/183771; and Konermann et al., Cell 173 (2018), 665-676, which are incorporated herein by reference in their entireties. Still other Cas mutants include eSpCas9 1.0 (K810A, K1003A, R1060A), eSpCas9 1.1 (K848A, K1003A, R1060A), SpCas9-HF1 (N497A, R661A, Q695A, Q926A), HypaCas9 (N692A, M694A, Q695A, H698A), EvoCas9 (M495V, Y515N, K526E, R661L), Sniper Cas9 (F539S, M7631, K890N), HiFi Cas9 V3 (R691A), Opti-SpCas9 (R661A and K1003H), and OptiHF-SpCas9 (Q695A, K848A, E293M, T924V and Q926A) (Slaymaker et al., Science 351, 84-88; Kleinstiver et al., Nature 523, 490-495; Chen et al. Nature 550, 407-410; Casini et al, Nature Biotechnology 36, 265-271: Lee et al., Nature Communications 9, 3048, Vakulskas et al., Nature Medicine 24, 1216-1224; Choi et al., Nature Methods 16, 722-730).

In some embodiments, the CRISPR nuclease can be a type II CRISPR/Cas 9 protein. For example, the CRISPR nuclease can be Streptococcus pyogenes Cas9 (SpCas9), Streptococcus thermophilus Cas9 (StCas9), Streptococcus pasteurianus (SpaCas9), Campylobacter jejuni Cas9 (CjCas9), Staphylococcus aureus (SaCas9), Francisella novicida Cas9 (FnCas9), Neisseria cinerea Cas9 (NcCas9), or Neisseria meningitis Cas9 (NmCas9). In other embodiments, the CRISPR nuclease can be a type V CRISPR/Cas12 (Cpf1) protein, e.g., Francisella novicida Cas12 (Cpf1) (FnCpf1), Acidaminococcus sp. Cas12 (Cpf1) (AsCpf1), or Lachnospiraceae bacterium ND2006 Cas12 (Cpf1) (LbCpf1). In further embodiments, the CRISPR nuclease can be a type VI CRISPR/Cas13 protein, e.g., Leptotrichia wadei Cas13a (LwaCas13a) or Leptotrichia shahii Cas13a (LshCas13a).

In general, the CRISPR nuclease comprises at least one nuclease domain having endonuclease activity. For example, a Cas9 nuclease comprises a HNH domain, which cleaves the guide RNA complementary strand, and a RuvC domain, which cleaves the non-complementary strand, a Cpf1 protein comprises a RuvC domain and a NUC domain, and a Cas13a nuclease comprises two HNEPN domains. In some embodiments, both nuclease domains are active and the CRISPR nuclease has double-stranded cleavage activity (i.e., cleaves both strands of a double-stranded nucleic acid sequence). In other embodiments, one of the nuclease domains is inactivated by one or more mutations and/or deletions, and the CRISPR variant is a nickase that cleaves one strand of a double-stranded nucleic acid sequence. For example, one or more mutations in the RuvC domain of Cas9 protein (e.g., D10A, D8A, E762A, and/or D986A) results in an HNH nickase that nicks the guide RNA complementary strand; and one or more mutations in the HNH domain of Cas9 protein (e.g., H840A, H559A, N854A, N856A, and/or N863A) results in a RuvC nickase that nicks the guide RNA non-complementary strand. Comparable mutations can convert Cas12/Cpf1 and Cas13a nucleases to nickases. Cas nickases can be sourced from the same bacterial species detailed above with respect to Cas nucleases.

In some embodiments, the CRISPR system (e.g., an RNP) is capable of making a site-specific base edit mediated by an C G to T A or an A T to G C base editing deaminase enzymes (Gaudelli et al., Programmable base editing of A T to G C in genomic DNA without DNA cleavage.” Nature 551, 464-471 (2017); Nishida et al. “Targeted nucleotide editing using hybrid prokaryotic and vertebrate adaptive immune systems.” Science 353 (6305) (2016); Komor et al. “Programmable editing of a target base in genomic DNA without double-stranded DNA cleavage.” Nature 533, 420-424 (2016)). Catalytically dead dCas9 fused to a cytidine deaminase or an adenine deaminase protein becomes a specific base editor that can alter DNA bases without inducing a DNA break. Base editors convert C->T (or G->A on the opposite strand) or an adenine base editor that would convert adenine to inosine, resulting in an A->G change within an editing window specified by the gRNA. As used herein, guide polynucleotide/Cas endonuclease complex, guide polynucleotide/Cas endonuclease system, guide polynucleotide/Cas complex, guide polynucleotide/Cas system, guided Cas system, and plural forms of the foregoing, are used interchangeably herein and refer to at least one guide polynucleotide and at least one Cas protein (i.e., endonuclease) that are capable of forming a complex, wherein said guide polynucleotide/Cas endonuclease complex can direct the Cas endonuclease to a DNA target site, enabling the Cas endonuclease to recognize, bind to, and optionally nick or cleave (introduce a single or double strand break) the DNA target site. A guide polynucleotide/Cas endonuclease complex herein can comprise Cas protein(s) and suitable polynucleotide component(s) of any of the four known CRISPR systems (Horvath and Barrangou, 2010, Science 327:167-170) such as a type I, II, or III CRISPR system. A Cas endonuclease unwinds the DNA duplex at the target sequence and optionally cleaves at least one DNA strand, as mediated by recognition of the target sequence by a polynucleotide (such as, but not limited to, a crRNA or guide RNA) that is in complex with the Cas protein. Such recognition and cutting of a target sequence by a Cas endonuclease typically occurs if the correct protospacer-adjacent motif (PAM) is located at or adjacent to the 3′ end or the 5′ end of the DNA target sequence. Alternatively, a Cas protein herein may lack DNA cleavage or nicking activity but can still specifically bind to a DNA target sequence when complexed with a suitable RNA component.

In some embodiments, the deaminase domain is fused to the N-terminus of the nuclease-inactivated Cas9 domain. In some embodiments, the deaminase domain is fused to the C-terminus of the nuclease-inactivated Cas9 domain. In some embodiments, the deaminase domain and the nuclease inactivated Cas9 domain are fused through a linker. The linker can be a non-functional amino acid sequence having no secondary or higher structure, N-terminus and one or more NLSs at the C-terminus. Where there are more than one NLS, each NLS may be selected as independent from other NLSs. In some embodiments, the targeted base-editing fusion protein comprises two NLSs, for example, the two NLSs are located at the N-terminus and the C-terminus, respectively.

In some embodiment, a targeted base modification is a conversion of any nucleotide C, A, T, or G, to any other nucleotide. Any one of a C, A, T or G nucleotide can be exchanged in a site-directed way as mediated by a base editor, or a catalytically active fragment thereof, to another nucleotide. A base editing complex can comprise any base editor, or a base editor domain or catalytically active fragment thereof, which can convert a nucleotide of interest into any other nucleotide of interest in a targeted way.

A base editing domain according to the present disclosure can comprise at least one cytidine deaminase, or a catalytically active fragment thereof. The at least one base editing complex can comprise the cytidine deaminase, or a domain thereof in the form of a catalytically active fragment, as base editor.

According to the present disclosure, cytidine deaminases that can be used in connection with the present disclosure include, but are not limited to, members of the enzyme family known as apolipoprotein B mRNA-editing complex (APOBEC) family deaminase, an activation-induced deaminase (AID), or a cytidine deaminase 1 (CDA1). In particular embodiments, the deaminase in an APOBEC1 deaminase, an APOBEC2 deaminase, an APOBEC3A deaminase, an APOBEC3B deaminase, an APOBEC3C deaminase, and APOBEC3D deaminase, an APOBEC3E deaminase, an APOBEC3F deaminase an APOBEC3G deaminase, an APOBEC3H deaminase, or an APOBEC4 deaminase.

The cytidine deaminase is capable of targeting cytosine in a DNA single strand. In certain example embodiments the cytodine deaminase may edit on a single strand present outside of the binding component e.g., bound Cas9 and/or Cas13. In other example embodiments, the cytodine deaminase may edit at a localized bubble, such as a localized bubble formed by a mismatch at the target edit site but the guide sequence.

In some embodiments, the cytidine deaminase protein recognizes and converts one or more target cytosine residue(s) in a single-stranded bubble of a DNA-RNA heteroduplex into uracil residues(s). In some embodiments, the cytidine deaminase protein recognizes a binding window on the single-stranded bubble of a DNA-RNA heteroduplex. In some embodiments, the binding window contains at least one target cytosine residue(s). In some embodiments, the binding window is in the range of about 3 bp to about 100 bp. In some embodiments, the binding window is in the range of about 5 bp to about 50 bp. In some embodiments, the binding window is in the range of about 10 bp to about 30 bp. In some embodiments, the binding window is about 1 bp, 2 bp, 3 bp, 5 bp, 7 bp, 10 bp, 15 bp, 20 bp, 25 bp, 30 bp, 40 bp, 45 bp, 50 bp, 55 bp, 60 bp, 65 bp, 70 bp, 75 bp, 80 bp, 85 bp, 90 bp, 95 bp, or 100 bp.

In some embodiments, the cytidine deaminase protein comprises one or more deaminase domains. Not intended to be bound by theory, it is contemplated that the deaminase domain functions to recognize and convert one or more target cytosine (C) residue(s) contained in a single-stranded bubble of a DNA-RNA heteroduplex into (an) uracil (U) residue(s). In some embodiments, the deaminase domain comprises an active center. In some embodiments, the active center comprises a zinc ion. In some embodiments, amino acid residues in or near the active center interact with one or more nucleotide(s) 5′ to a target cytosine residue. In some embodiments, amino acid residues in or near the active center interact with one or more nucleotide(s) 3′ to a target cytosine residue.

In some embodiments, the cytidine deaminase comprises human APOBEC1 full protein (hAPOBECI) or the deaminase domain thereof (hAPOBECI-D) or a C-terminally truncated version thereof (hAPOBEC-T). In some embodiments, the cytidine deaminase is an APOBEC family member that is homologous to hAPOBECI, hAPOBEC-D or hAPOBEC-T. In some embodiments, the cytidine deaminase comprises human AID1 full protein (hAID) or the deaminase domain thereof (hAID-D) or a C-terminally truncated version thereof (hAID-T). In some embodiments, the cytidine deaminase is an AID family member that is homologous to hAID, hAID-D or hAID-T. In some embodiments, the hAIDT is a hAID which is C-terminally truncated by about 20 amino acids.

In some embodiments, the cytidine deaminase comprises the wild-type amino acid sequence of a cytosine deaminase. In some embodiments, the cytidine deaminase comprises one or more mutations in the cytosine deaminase sequence, such that the editing efficiency, and/or substrate editing preference of the cytosine deaminase is changed according to specific needs.

Certain mutations of APOBEC1 and APOBEC3 proteins have been described in Kim et al., Nature Biotechnology (2017) 35 (4): 371-377 and Harris et al. Mol. Cell (2002) 10:1247-1253, each of which is incorporated herein by reference in its entirety.

Additional embodiments of the cytidine deaminase are disclosed in WO2017/070632 and WO2018/213726, each of which is incorporated herein by reference in its entirety.

Zinc Finger Nucleases. In still other embodiments, the programmable DNA modification protein having nuclease activity can be a pair of zinc finger nucleases (ZFN). A ZFN comprises a DNA binding zinc finger region and a nuclease domain. The zinc finger region can comprise from about two to seven zinc fingers, for example, about four to six zinc fingers, wherein each zinc finger binds three consecutive base pairs. The zinc finger region can be engineered to recognize and bind to any DNA sequence. Zinc finger design tools or algorithms are available on the internet or from commercial sources. The zinc fingers can be linked together using suitable linker sequences.

A ZFN also comprises a nuclease domain, which can be obtained from any endonuclease or exonuclease. Non-limiting examples of endonucleases from which a nuclease domain can be derived include, but are not limited to, restriction endonucleases and homing endonucleases. In some embodiments, the nuclease domain can be derived from a type II-S restriction endonuclease. Type II-S endonucleases cleave DNA at sites that are typically several base pairs away from the recognition/binding site and, as such, have separable binding and cleavage domains. These enzymes generally are monomers that transiently associate to form dimers to cleave each strand of DNA at staggered locations. Non-limiting examples of suitable type II-S endonucleases include Bfil, Bpml, Bsal, Bsgl, BsmBI, Bsml, BspMI, Fokl, Mboll, and Sapl. In some embodiments, the nuclease domain can be a Fokl nuclease domain or a derivative thereof. The type II-S nuclease domain can be modified to facilitate dimerization of two different nuclease domains. For example, the cleavage domain of Fokl can be modified by mutating certain amino acid residues. By way of non-limiting example, amino acid residues at positions 446, 447, 479, 483, 484, 486, 487, 490, 491, 496, 498, 499, 500, 531, 534, 537, and 538 of Fokl nuclease domains are targets for modification. In specific embodiments, the Fokl nuclease domain can comprise a first Fokl half-domain comprising Q486E, 1499L, and/or N496D mutations, and a second Fokl half-domain comprising E490K, 1538K, and/or H537R mutations. In some embodiments, the ZFN has double-stranded cleavage activity. In other embodiments, the ZFN has nickase activity (i.e., one of the nuclease domains has been inactivated).

Transcription Activator-like Effector Nucleases. In alternate embodiments, the programmable DNA modification protein having nuclease activity can be a transcription activator-like effector nuclease (TALEN). TALENs comprise a DNA binding domain composed of highly conserved repeats derived from transcription activator-like effectors (TALEs) that is linked to a nuclease domain. TALEs are proteins secreted by plant pathogen Xanthomonas to alter transcription of genes in host plant cells. TALE repeat arrays can be engineered via modular protein design to target any DNA sequence of interest. The nuclease domain of TALENs can be any nuclease domain as described above in the subsection describing ZFNs. In specific embodiments, the nuclease domain is derived from Fokl (Sanjana et al., 2012, Nat Protoc, 7 (1): 171-192). The TALEN can have double-stranded cleavage activity or nickase activity.

Meganucleases or Rare-Cutting Endonucleases. In still other embodiments, the programmable DNA modification protein having nuclease activity can be a meganuclease or derivative thereof. Meganucleases are endodeoxyribonucleases characterized by long recognition sequences, i.e., the recognition sequence generally ranges from about 12 base pairs to about 45 base pairs. As a consequence of this requirement, the recognition sequence generally occurs only once in any given genome. Among meganucleases, the family of homing endonucleases named LAGLIDADG (SEQ ID NO: 1) has become a valuable tool for the study of genomes and genome engineering. In some embodiments, the meganuclease can be I-Scel, I-Tevl, or variants thereof. A meganuclease can be targeted to a specific chromosomal sequence by modifying its recognition sequence using techniques well known to those skilled in the art. In alternate embodiments, the programmable DNA modification protein having nuclease activity can be a rare-cutting endonuclease or derivative thereof. Rare-cutting endonucleases are site-specific endonucleases whose recognition sequence occurs rarely in a genome, preferably only once in a genome. The rare-cutting endonuclease may recognize a 7-nucleotide sequence, an 8-nucleotide sequence, or longer recognition sequence. Non-limiting examples of rare-cutting endonucleases include NotI, AscI, PacI, AsiSI, SbfI, and FseI.

Programmable DNA Binding Domains Linked to Nuclease Domains. In yet additional embodiments, the programmable DNA modification protein having nuclease activity can be a chimeric protein comprising a programmable DNA binding domain linked to a nuclease domain. The nuclease domain can be any of those described above in the subsection describing ZFNs (e.g., the nuclease domain can be a Fokl nuclease domain), a nuclease domain derived from a CRISPR nuclease (e.g., RuvC or HNH nuclease domains of Cas9), or a nuclease domain derived from a meganuclease or rare-cutting endonuclease.

The programmable DNA binding domain of the chimeric protein can be any programmable DNA binding protein such as, e.g., a zinc finger protein or a transcription activator-like effector. Alternatively, the programmable DNA binding domain can be a catalytically inactive (dead) CRISPR protein that was modified by deletion or mutation to lack all nuclease activity. For example, the catalytically inactive CRISPR protein can be a catalytically inactive (dead) Cas9 (dCas9) in which the RuvC domain comprises a D10A, D8A, E762A, and/or D986A mutation and the HNH domain comprises a H840A, H559A, N854A, N865A, and/or N863A mutation. Alternatively, the catalytically inactive CRISPR protein can be a catalytically inactive (dead) Cpf1 protein comprising comparable mutations in the nuclease domains. In still other embodiments, the programmable DNA binding domain can be a catalytically inactive meganuclease in which nuclease activity was eliminated by mutation and/or deletion, e.g., the catalytically inactive meganuclease can comprise a C-terminal truncation.

Programmable DNA Modification Proteins with Non-Nuclease Activity

In alternate embodiments, the programmable DNA modification protein can be a chimeric protein comprising a programmable DNA binding domain linked to a non-nuclease domain. The programmable DNA binding domain can be a zinc finger protein, a transcription activator-like effector, a catalytically inactive (dead) CRISPR protein, or a catalytically inactive (dead) meganuclease. For example, the catalytically inactive CRISPR protein can be a catalytically inactive (dead) Cas9 (dCas9) in which the RuvC domain comprises a D10A, D8A, E762A, and/or D986A mutation and the HNH domain comprises a H840A, H559A, N854A, N865A, and/or N863A mutation. Alternatively, the catalytically inactive CRISPR protein can be a catalytically inactive (dead) Cpf1 protein comprising comparable mutations in the nuclease domains.

In some embodiments, the non-nuclease domain of the chimeric protein can be an epigenetic modification domain, which alters DNA or chromatin structure (and may or may not alter DNA sequence). Non-limiting examples of suitable epigenetic modification domains include those with DNA methyltransferase activity (e.g., cytosine methyltransferase), DNA demethylase activity, DNA deamination (e.g., cytosine deaminase, adenosine deaminase, guanine deaminase), DNA amination, DNA oxidation activity, DNA helicase activity, histone acetyltransferase (HAT) activity (e.g., HAT domain derived from E1A binding protein p300), histone deacetylase activity, histone methyltransferase activity, histone demethylase activity, histone kinase activity, histone phosphatase activity, histone ubiquitin ligase activity, histone deubiquitinating activity, histone adenylation activity, histone deadenylation activity, histone SUMOylating activity, histone deSUMOylating activity, histone ribosylation activity, histone deribosylation activity, histone myristoylation activity, histone demyristoylation activity, histone citrullination activity, histone alkylation activity, histone dealkylation activity, or histone oxidation activity. In specific embodiments, the epigenetic modification domain can comprise cytidine deaminase activity, histone acetyltransferase activity, or DNA methyltransferase activity.

In other embodiments, the non-nuclease modification domain of the chimeric protein can be a transcriptional activation domain or transcriptional repressor domain. Suitable transcriptional activation domains include, without limit, herpes simplex virus VP16 domain, VP64 (which is a tetrameric derivative of VP16), VP160, NFκB p65 activation domains, p53 activation domains 1 and 2, CREB (CAMP response element binding protein) activation domains, E2A activation domains, activation domain from human heat-shock factor 1 (HSF1), or NFAT (nuclear factor of activated T-cells) activation domains. Non-limiting examples of suitable transcriptional repressor domains include inducible cAMP early repressor (ICER) domains, Kruppel-associated box (KRAB) repressor domains, YY1 glycine rich repressor domains, Sp1-like repressors, E(spl) repressors, IκB repressor, or methyl-CpG binding protein 2 (MeCP2) repressors. Transcriptional activation or transcriptional repressor domains can be genetically fused to the DNA binding protein or bound via noncovalent protein-protein, protein-RNA, or protein-DNA interactions.

In particular embodiments, the non-nuclease domain of the chimeric protein can comprise cytidine deaminase activity, histone acetyltransferase activity, transcriptional activation activity, or transcriptional repressor activity.

In some embodiments, the chimeric protein having non-nuclease activity can further comprise at least one detectable label. The detectable label can be a fluorophore (e.g., FAM, TMR, Cy3, Cy5, Texas Red, Oregon Green, Alexa Fluors, Halo tags, or suitable fluorescent dye), a detection tag (e.g., biotin, digoxigenin, and the like), quantum dots, or gold particles.

Optional Nuclear Localization Signal, Cell-Penetrating Domain, and/or Marker Domain

The programmable DNA modification proteins (e.g., CRISPR-Cas) disclosed herein can further comprise at least one nuclear localization signal, cell-penetrating domain, and/or marker domain.

Non-limiting examples of nuclear localization signals include PKKKRKV (SEQ ID NO:2), PKKKRRV (SEQ ID NO:3), KRPAATKKAGQAKKKK (SEQ ID NO:4), YGRKKRRQRRR (SEQ ID NO:5), RKKRRQRRR (SEQ ID NO:6), PAAKRVKLD (SEQ ID NO:7), RQRRNELKRSP (SEQ ID NO:8), VSRKRPRP (SEQ ID NO:9), PPKKARED (SEQ ID NO:10), PQPKKKPL (SEQ ID NO:11), SALIKKKKKMAP (SEQ ID NO: 12), PKQKKRK (SEQ ID NO: 13), RKLKKKIKKL (SEQ ID NO: 14), REKKKFLKRR (SEQ ID NO: 15), KRKGDEVDGVDEVAKKKSKK (SEQ ID NO: 16), RKCLQAGMNLEARKTKK (SEQ ID NO: 17), NQSSNFGPMKGGNFGGRSSGPYGGGGQYFAKPRNQGGY (SEQ ID NO: 18), and RMRIZFKNKGKDTAELRRRRVEVSVELRKAKKDEQILKRRNV (SEQ ID NO:19).

Examples of suitable cell-penetrating domains include, without limit, GRKKRRQRRRPPQPKKKRKV (SEQ ID NO:20), PLSSIFSRIGDPPKKKRKV (SEQ ID NO:21), GALFLGWLGAAGSTMGAPKKKRKV (SEQ ID NO:22), GALFLGFLGAAGSTMGAWSQPKKKRKV (SEQ ID NO:23), KETWWETWWTEWSQPKKKRKV (SEQ ID NO:24), YARAAARQARA (SEQ ID NO: 25), THRLPRRRRRR (SEQ ID NO:26), GGRRARRRRRR (SEQ ID NO: 27), RRQRRTSKLMKR (SEQ ID NO:28), GWTLNSAGYLLGKINLKALAALAKKIL (SEQ ID NO:29), KALAWEAKLAKALAKALAKHLAKALAKALKCEA (SEQ ID NO:30), and RQIKIWFQNRRMKWKK (SEQ ID NO:31).

Marker domains include fluorescent proteins and purification or epitope tags. Suitable fluorescent proteins include, without limit, green fluorescent proteins (e.g., GFP, eGFP, GFP-2, tagGFP, turboGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, ZsYellow1), blue fluorescent proteins (e.g., BFP, EBFP, EBFP2, Azurite, mKalama1, GFPuv, Sapphire, T-sapphire), cyan fluorescent proteins (e.g., ECFP, Cerulean, CyPet, AmCyan1, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (e.g., mOrange, mKO, Kusabira-Orange, Monomeric Kusabira-Orange, mTangerine, tdTomato). Non-limiting examples of suitable purification or epitope tags include 6×His (SEQ ID NO: 32), FLAG®, HA, GST, Myc, and the like.

The at least one nuclear localization signal, cell-penetrating domain, and/or marker domain can be located at the N-terminus, the C-terminus, and/or in an internal location of the fusion protein.

In specific embodiments, the programmable DNA modification protein of the fusion protein is a CRISPR protein. For example, the CRISPR protein can be Streptococcus pyogenes Cas9 (SpCas9), Streptococcus thermophilus Cas9 (StCas9), Streptococcus pasteurianus (SpaCas9), Campylobacter jejuni Cas9 (CjCas9), Staphylococcus aureus (SaCas9), Francisella novicida Cas9 (FnCas9), Neisseria cinerea Cas9 (NcCas9), Neisseria meningitis Cas9 (NmCas9), Francisella novicida Cpf1 (FnCpf1), Acidaminococcus sp. Cpf1 (AsCpf1), or Lachnospiraceae bacterium ND2006 Cpf1 (LbCpf1).

### Complexes

Another aspect of the present disclosure encompasses complexes comprising at least one CRISPR system (i.e., CRISPR protein and guide RNA) and, optionally, at least one nucleosome interacting protein domain. In some embodiments, the at least one nucleosome interacting protein domain can be linked to the CRISPR protein of the CRISPR system (i.e., the complex comprises a CRISPR fusion protein as described above). In other embodiments, the at least one nucleosome interacting protein domain can be linked to the guide RNA of the CRISPR system. The linkage can be direct or indirect, essentially as described above. For example, a nucleosome interacting protein domain can be linked to an RNA aptamer binding protein, and the guide RNA can comprise aptamer sequences, such that binding of the RNA aptamer binding protein to the RNA aptamer sequence links the nucleosome interacting protein domain to the guide RNA.

Nucleosome interacting protein domains are described above in section (I) (a), and CRISPR proteins are detailed above. The CRISPR protein can have nuclease or nickase activity (e.g., can be a type II CRISPR/Cas9, type V CRISPR/Cpf1, or type VI CRISPR/Cas13). For example, a complex can comprise a CRISPR nuclease, or a complex can comprise two CRISPR nickases. Alternatively, the CRISPR protein can be modified to lack all nuclease activity and linked to non-nuclease domains (e.g., domains having cytosine deaminase activity, histone acetyltransferase activity, transcriptional activation activity, or transcriptional repressor activity). In some embodiments, the non-nuclease domain also can be linked to an RNA aptamer binding protein.

Regardless of whether a nucleosome interacting protein domain is present or absent, in the CRISPR systems described herein a guide RNA comprises (i) a CRISPR RNA (crRNA) that contains a guide sequence at the 5′ end that hybridizes with a target sequence and (ii) a transacting crRNA (tracrRNA) sequence that interacts with the CRISPR protein. The crRNA guide sequence of each guide RNA is different (i.e., is sequence specific). The tracrRNA sequence is generally the same in guide RNAs designed to complex with a CRISPR protein from a particular bacterial species.

The crRNA guide sequence is designed to hybridize with a target sequence (i.e., protospacer) that is bordered by a protospacer adjacent motif (PAM) in a double-stranded sequence. PAM sequences for Cas9 proteins include 5′-NGG, 5′-NGGNG, 5′-NNAGAAW, and 5′-ACAY, and PAM sequences for Cpf1 include 5′-TTN (wherein N is defined as any nucleotide, W is defined as either A or T, and Y is defined as either C or T). In general, the complementarity between the crRNA guide sequence and the target sequence is at least 80%, at least 85%, at least 90%, at least 95%, or at least 99%. In specific embodiments, the complementarity is complete (i.e., 100%). In various embodiments, the length of the crRNA guide sequence can range from about 15 nucleotides to about 25 nucleotides. For example, the crRNA guide sequence can be about 15, 16, 17, 18, 19, 20, 21, 22, 23, 24, or 25 nucleotides in length. In specific embodiments, the crRNA can be about 19, 20, 21, or 22 nucleotides in length.

The crRNA and tracrRNA comprise repeat sequences that form one or more one-stem loop structures, which can interact with the CRISPR protein. The length of each loop and stem can vary. For example, the one or more loops can range from about 3 to about 10 nucleotides in length, and the one or more stems can range from about 6 to about 20 base pairs in length. The one or more stems can comprise one or more bulges of 1 to about 10 nucleotides.

The crRNA can range in length from about 25 nucleotides to about 100 nucleotides. In various embodiments, the crRNA can range in length from about 25 to about 50 nucleotides, from about 50 to about 75 nucleotides, or from about 75 to about 100 nucleotides. The tracrRNA can range in length from about 50 nucleotides to about 300 nucleotides. In various embodiments, the tracrRNA can range in length from about 50 to about 90 nucleotides, from about 90 to about 110 nucleotides, from about 110 to about 130 nucleotides, from about 130 to about 150 nucleotides, from about 150 to about 170 nucleotides, from about 170 to about 200 nucleotides, from about 200 to about 250 nucleotides, or from about 250 to about 300 nucleotides.

The tracrRNA sequence in the guide RNA generally is based upon the coding sequence of wild type tracrRNA in the bacterial species of interest. In some embodiments, the wild-type tracrRNA sequence (or the crRNA constant repeat region and the corresponding 5′ region of the tracrRNA that forms a duplex structure with the crRNA constant repeat region) can be modified to facilitate secondary structure formation, increase secondary structure stability, facilitate expression in eukaryotic cells, increase editing efficiency, and so forth. For example, one or more nucleotide changes can be introduced into the constant guide RNA sequence.

The guide RNA can be a single molecule (i.e., a single guide RNA or sgRNA), wherein the crRNA sequence is linked to the tracrRNA sequence. Alternatively, the guide RNA can be two separate molecules. A first molecule comprising the crRNA guide sequence at the 5′ end and additional sequence at 3′ end that is capable of base pairing with the 5′ end of a second molecule, wherein the second molecule comprises 5′ sequence that is capable of base pairing with the 3′ end of the first molecule, as well as additional tracrRNA sequence. In some embodiments, the guide RNA of type V CRISPR/Cpf1 systems can comprise only crRNA.

In some embodiments, the one or more stem-loop regions of the guide RNA can be modified to comprise one or more aptamer sequences (Konermann et al., Nature, 2015, 517 (7536): 583-588; Zalatan et al., Cell, 2015, 160 (1-2): 339-50). Examples of suitable RNA aptamer protein domains include MS2 coat protein (MCP), PP7 bacteriophage coat protein (PCP), Mu bacteriophage Com protein, lambda bacteriophage N22 protein, stem-loop binding protein (SLBP), Fragile X mental retardation syndrome-related protein 1 (FXR1), proteins derived from bacteriophage such as AP205, BZ13, f1, f2, fd, fr, ID2, JP34/GA, JP501, JP34, JP500, KU1, M11, M12, MX1, NL95, PP7, ϕCb5, ϕCb8r, ϕCb12r, ϕCb23r, Qβ, R17, SP-β, TW18, TW19, and VK, fragments thereof, or derivatives thereof. The length of the additional aptamer sequence can range from about 20 nucleotides to about 200 nucleotides.

The guide RNA can comprise standard ribonucleotides, modified ribonucleotides (e.g., pseudouridine), ribonucleotide isomers, and/or ribonucleotide analogs. In some embodiments, the guide RNA can further comprise at least one detectable label. The detectable label can be a fluorophore (e.g., FAM, TMR, Cy3, Cy5, Texas Red, Oregon Green, Alexa Fluors, Halo tags, or suitable fluorescent dye), a detection tag (e.g., biotin, digoxigenin, and the like), quantum dots, or gold particles. Those skilled in the art are familiar with gRNA design and construction, e.g., gRNA design tools are available on the internet or from commercial sources.

The guide RNA can be synthesized chemically, synthesized enzymatically, or a combination thereof. For example, the guide RNA can be synthesized using standard phosphoramidite-based solid-phase synthesis methods. Alternatively, the guide RNA can be synthesized in vitro by operably linking DNA encoding the guide RNA to a promoter control sequence that is recognized by a phage RNA polymerase. Examples of suitable phage promoter sequences include T7, T3, SP6 promoter sequences, or variations thereof. In embodiments in which the guide RNA comprises two separate molecules (i.e., crRNA and tracrRNA), the crRNA can be chemically synthesized and the tracrRNA can be enzymatically synthesized.

In some embodiments, the complex may further comprise a donor polynucleotide comprising a donor sequence that is flanked by sequence having substantial sequence identity to sequences located on either side of the target chromosomal sequence, such that during repair of the double-stranded break by a homology directed repair process (HDR) the donor sequence in the donor polynucleotide can be exchanged with or integrated into the chromosomal sequence at the target chromosomal sequence. Integration of an exogenous sequence is termed a “knock in.”

In embodiments in which the programmable DNA modification protein comprises nuclease activity, the method can further comprise introducing at least one donor polynucleotide into the cell via electroporation as described herein and utilizing the electroporation enhancers. The donor polynucleotide can be single-stranded or double-stranded, linear or circular, and/or RNA or DNA. In some embodiments, the donor polynucleotide can be a vector, e.g., a plasmid vector.

The donor polynucleotide comprises at least one donor sequence. In some embodiments, the donor sequence of the donor polynucleotide can be a modified version of an endogenous or native chromosomal sequence. For example, the donor sequence can be essentially identical to a portion of the chromosomal sequence at or near the sequence targeted by the DNA modification protein, but which comprises at least one nucleotide change. Thus, upon integration or exchange with the native sequence, the sequence at the targeted chromosomal location comprises at least one nucleotide change. For example, the change can be an insertion of one or more nucleotides, a deletion of one or more nucleotides, a substitution of one or more nucleotides, or combinations thereof. As a consequence of the “gene correction” integration of the modified sequence, the cell can produce a modified gene product from the targeted chromosomal sequence.

In other embodiments, the donor sequence of the donor polynucleotide can be an exogenous sequence. As used herein, an “exogenous” sequence refers to a sequence that is not native to the cell, or a sequence whose native location is in a different location in the genome of the cell. For example, the exogenous sequence can comprise protein coding sequence, which can be operably linked to an exogenous promoter control sequence such that, upon integration into the genome, the cell is able to express the protein coded by the integrated sequence. Alternatively, the exogenous sequence can be integrated into the chromosomal sequence such that its expression is regulated by an endogenous promoter control sequence. In other iterations, the exogenous sequence can be a transcriptional control sequence, another expression control sequence, an RNA coding sequence, and so forth. As noted above, integration of an exogenous sequence into a chromosomal sequence is termed a “knock in.”

As can be appreciated by those skilled in the art, the length of the donor sequence can and will vary. For example, the donor sequence can vary in length from several nucleotides to hundreds of nucleotides to hundreds of thousands of nucleotides.

Typically, the donor sequence in the donor polynucleotide is flanked by an upstream sequence and a downstream sequence, which have substantial sequence identity to sequences located upstream and downstream, respectively, of the sequence targeted by the programmable DNA modification protein. Because of these sequence similarities, the upstream and downstream sequences of the donor polynucleotide permit homologous recombination between the donor polynucleotide and the targeted chromosomal sequence such that the donor sequence can be integrated into (or exchanged with) the chromosomal sequence.

The upstream sequence, as used herein, refers to a nucleic acid sequence that shares substantial sequence identity with a chromosomal sequence upstream of the sequence targeted by the programmable DNA modification protein. Similarly, the downstream sequence refers to a nucleic acid sequence that shares substantial sequence identity with a chromosomal sequence downstream of the sequence targeted by the programmable DNA modification protein. As used herein, the phrase “substantial sequence identity” refers to sequences having at least about 75% sequence identity. Thus, the upstream and downstream sequences in the donor polynucleotide can have about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with sequence upstream or downstream to the target sequence. In an exemplary embodiment, the upstream and downstream sequences in the donor polynucleotide can have about 95% or 100% sequence identity with chromosomal sequences upstream or downstream to the sequence targeted by the programmable DNA modification protein.

In some embodiments, the upstream sequence shares substantial sequence identity with a chromosomal sequence located immediately upstream of the sequence targeted by the programmable DNA modification protein. In other embodiments, the upstream sequence shares substantial sequence identity with a chromosomal sequence that is located within about one hundred (100) nucleotides upstream from the target sequence. Thus, for example, the upstream sequence can share substantial sequence identity with a chromosomal sequence that is located about 1 to about 20, about 21 to about 40, about 41 to about 60, about 61 to about 80, or about 81 to about 100 nucleotides upstream from the target sequence. In some embodiments, the downstream sequence shares substantial sequence identity with a chromosomal sequence located immediately downstream of the sequence targeted by the programmable DNA modification protein. In other embodiments, the downstream sequence shares substantial sequence identity with a chromosomal sequence that is located within about one hundred (100) nucleotides downstream from the target sequence. Thus, for example, the downstream sequence can share substantial sequence identity with a chromosomal sequence that is located about 1 to about 20, about 21 to about 40, about 41 to about 60, about 61 to about 80, or about 81 to about 100 nucleotides downstream from the target sequence.

Each upstream or downstream sequence can range in length from about 20 nucleotides to about 5000 nucleotides. In some embodiments, upstream and downstream sequences can comprise about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2800, 3000, 3200, 3400, 3600, 3800, 4000, 4200, 4400, 4600, 4800, or 5000 nucleotides. In specific embodiments, upstream and downstream sequences can range in length from about 50 to about 1500 nucleotides.

### Cell Types

A variety of cells are suitable for use in the methods, compositions, and uses disclosed herein. Preferably, the cell is a eukaryotic cell that is transfected in accordance with the disclosure (that is, transfected via electroporation in the presence of a polyanionic polymer (e.g., dextran sulfate)). For example, the cell can be a human cell, a non-human mammalian cell, a non-mammalian vertebrate cell, an invertebrate cell, an insect cell, a plant cell, a yeast cell, or a single cell eukaryotic organism. In some embodiments, the cell can also be a one cell embryo. For example, a non-human mammalian embryo including rat, hamster, rodent, rabbit, feline, canine, ovine, porcine, bovine, equine, and primate embryos. In still other embodiments, the cell can be a stem cell such as embryonic stem cells, ES-like stem cells, fetal stem cells, adult stem cells, and the like. In one embodiment, the stem cell is not a human embryonic stem cell. Furthermore, the stem cells may include those made by the techniques disclosed in WO 2003/046141, which is incorporated herein in its entirety, or Chung et al. (Cell Stem Cell, 2008, 2:113-117). The cell can be in vitro or in vivo (i.e., within an organism). In exemplary embodiments, the cell is a mammalian cell or mammalian cell line. In particular embodiments, the cell is a human cell or human cell line. In other particular embodiments, the cell is a plant cell or plant cell line.

In some embodiments, the disclosure provides methods, compositions, and uses as herein discussed, wherein the non-human mammal cell may be including, but not limited to, primate, bovine, ovine, procine, canine, rodent, Leporidae such as monkey, cow, sheep, pig, dog, rabbit, rat, or mouse cell. In some embodiments, the disclosure provides methods, compositions, and uses as herein discussed, wherein the cell may be a non-mammalian eukaryotic cell such as poultry bird (e.g., chicken), vertebrate fish (e.g., salmon) or shellfish (e.g., oyster, claim, lobster, shrimp) cell. In some embodiments, the disclosure provides methods, compositions, and uses as herein discussed, wherein the non-human eukaryote cell is a plant cell. The plant cell may be of a monocot or dicot or of a crop or grain plant such as cassava, corn, sorghum, soybean, wheat, oat, or rice. The plant cell may also be of an algae, tree or production plant, fruit or vegetable (e.g., trees such as citrus trees, e.g., orange, grapefruit or lemon trees; peach or nectarine trees; apple or pear trees; nut trees such as almond or walnut or pistachio trees; nightshade plants; plants of the genus Brassica; plants of the genus Lactuca; plants of the genus Spinacia; plants of the genus Capsicum; cotton, tobacco, asparagus, carrot, cabbage, broccoli, cauliflower, tomato, eggplant, pepper, lettuce, spinach, strawberry, blueberry, raspberry, blackberry, grape, coffee, cocoa, etc.).

In certain embodiments, the eukaryotic cell is a non-human animal (cell), such as (a cell or cell population of a) non-human mammal, non-human primate, an ungulate, rodent (preferably a mouse or rat), rabbit, canine, dog, cow, bovine, sheep, ovine, goat, pig, fowl, poultry, chicken, fish, insect, or arthropod, preferably a mammal, such as a rodent, in particular a mouse. In some embodiments, the organism or subject or cell may be (a cell or cell population derived from) an arthropod, for example, an insect, or a nematode. In some methods of the disclosure the organism or subject or cell is a plant (cell). In some embodiments, the organism or subject or cell is (or is derived from) algae, including microalgae, or fungus. One of skill in the art will appreciate that the eukaryotic cells which may be transplanted or introduced in a non-human eukaryote according to the methods as referred to herein are preferably derived from or originate from the same species as the eukaryote to which they are transplanted. For example, mouse cells can be transplanted in a mouse. In certain embodiments, the eukaryotic cell is an immunocompromised eukaryote, i.e., a eukaryote in which the immune system is partially or completely shut down. For instance, immunocompromised mice may be involved in the methods, compositions, and uses as described herein. Examples of immunocompromised mice include, but are not limited to Nude mice, RAG−/−mice, SCID (severe compromised immunodeficiency) mice, SCID-Beige mice, NOD (non-obese diabetic)-SCID mice, NOG or NSG mice, etc.

Non-limiting examples of suitable mammalian cells or cell lines include human embryonic kidney cells (HEK293, HEK293T); human cervical carcinoma cells (HELA); human lung cells (W138); human liver cells (Hep G2); human U2-OS osteosarcoma cells, human A549 cells, human A-431 cells, and human K562 cells; Chinese hamster ovary (CHO) cells, baby hamster kidney (BHK) cells; mouse myeloma NS0 cells, mouse embryonic fibroblast 3T3 cells (NIH3T3), mouse B lymphoma A20 cells; mouse melanoma B16 cells; mouse myoblast C2C12 cells; mouse myeloma SP2/0 cells; mouse embryonic mesenchymal C3H-10T1/2 cells; mouse carcinoma CT26 cells, mouse prostate DuCuP cells; mouse breast EMT6 cells; mouse hepatoma Hepa1c1c7 cells; mouse myeloma J5582 cells; mouse epithelial MTD-1A cells; mouse myocardial MyEnd cells; mouse renal RenCa cells; mouse pancreatic RIN-5F cells; mouse melanoma X64 cells; mouse lymphoma YAC-1 cells; rat glioblastoma 9L cells; rat B lymphoma RBL cells; rat neuroblastoma B35 cells; rat hepatoma cells (HTC); buffalo rat liver BRL 3A cells; canine kidney cells (MDCK); canine mammary (CMT) cells; rat osteosarcoma D17 cells; rat monocyte/macrophage DH82 cells; monkey kidney SV-40 transformed fibroblast (COS7) cells; monkey kidney CVI-76 cells; African green monkey kidney (VERO-76) cells. Still other non-limiting examples of suitable cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, C1R, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO—IR, CHO-K1, CHO-K2, CHO-T, CHO Dhfr−/−, COR-L23. COR-L23/CPR, COR-L23/5010, COR-L23/R23, COS-7. COV-434, CML T1, CMT, CT26, D17, DH82, DU145, DuCaP, EL4, EM2, EM3, EMT6/AR1, EMT6/AR10.0. FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells. Sf-9. SkBr3, T2, T-47D, T84, THP1 cell line, U373, U87, U937, VCaP, Vero cells, WM39, WT-49, X63, YAC-1, YAR, and transgenic varieties thereof. An extensive list of mammalian cell lines may be found in the American Type Culture Collection catalog (ATCC, Manassas, VA).

### Applications

The methods, compositions, uses, and/or kits disclosed herein can be used in a variety of therapeutic, diagnostic, industrial, and research applications. In some embodiments, the present disclosure can be used to modify any chromosomal sequence of interest in a cell, animal, or plant in order to model and/or study the function of genes, study genetic or epigenetic conditions of interest, or study biochemical pathways involved in various diseases or disorders. For example, transgenic organisms can be created that model diseases or disorders, wherein the expression of one or more nucleic acid sequences associated with a disease or disorder is altered. The disease model can be used to study the effects of mutations on the organism, study the development and/or progression of the disease, study the effect of a pharmaceutically active compound on the disease, and/or assess the efficacy of a potential gene therapy strategy.

In other embodiments, the methods, compositions, uses, and/or kits can be used to perform efficient and cost effective functional genomic screens, which can be used to study the function of genes involved in a particular biological process and how any alteration in gene expression can affect the biological process, or to perform saturating or deep scanning mutagenesis of genomic loci in conjunction with a cellular phenotype. Saturating or deep scanning mutagenesis can be used to determine critical minimal features and discrete vulnerabilities of functional elements required for gene expression, drug resistance, and reversal of disease, for example.

In further embodiments, the methods, compositions, uses, and/or kits disclosed herein can be used for diagnostic tests to establish the presence of a disease or disorder and/or for use in determining treatment options. Examples of suitable diagnostic tests include detection of specific mutations in cancer cells (e.g., specific mutation in EGFR, HER2, and the like), detection of specific mutations associated with particular diseases (e.g., trinucleotide repeats, mutations in β-globin associated with sickle cell disease, specific SNPs, etc.), detection of hepatitis, detection of viruses (e.g., Zika), and so forth.

In additional embodiments, the methods, compositions, uses, and/or kits disclosed herein can be used to correct genetic mutations associated with a particular disease or disorder such as, e.g., correct globin gene mutations associated with sickle cell disease or thalassemia, correct mutations in the adenosine deaminase gene associated with severe combined immune deficiency (SCID), reduce the expression of HTT, the disease-causing gene of Huntington's disease, or correct mutations in the rhodopsin gene for the treatment of retinitis pigmentosa. Such modifications may be made in cells ex vivo.

In still other embodiments, the methods, compositions, uses, and/or kits disclosed herein can be used to generate crop plants with improved traits or increased resistance to environmental stresses. The present disclosure can also be used to generate farm animals with improved traits or production animals. For example, pigs have many features that make them attractive as biomedical models, especially in regenerative medicine or xenotransplantation.

### Kits

A further aspect of the present disclosure provides kits comprising one or more components for use in the methods and uses described herein.

The kits can comprise, for instance, programmable DNA modification proteins, guide RNAs, transfection reagents, cell growth media, selection media, transcription reagents, nucleic acid purification reagents, protein purification reagents, buffers, solutions, and the like. In one particular embodiment, the kit comprises elements including a Cas protein and a guide RNA (which components may be provided separately or complexed together as a ribonucleoprotein) and a polyanionic polymer as described herein (e.g., dextran sulfate). The kits can additionally or alternatively comprise, for instance, cells, transfection reagents (e.g., electroporation solutions or components thereof), cell growth media, selection media, transcription reagents, nucleic acid purification reagents, protein purification reagents, buffers, solutions, and the like. Elements may be provided individually or in combinations, and may be provided in any suitable container, such as a vial, a bottle, or a tube.

In some embodiments, a kit comprises one or more reagents for use in a method utilizing one or more of the elements described herein. Reagents may be provided in any suitable container. For example, a kit may provide one or more reaction, reconstitution, stabilization, dilution, and/or storage buffers or solutions. Reagents may be provided in a form that is usable in a particular assay, or in a form that requires addition of one or more other components before use (e.g., in concentrate or lyophilized form). A buffer or solution can be any buffer or solution commonly used in biotechnology applications, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more oligonucleotides corresponding to a guide sequence for insertion into a vector so as to operably link the guide sequence and a regulatory element. In some embodiments, the kit comprises a homologous recombination template polynucleotide. In some embodiments, the kit comprises one or more of the vectors and/or one or more of the polynucleotides described herein. The kit may advantageously allow to provide all elements of the methods and/or systems of the disclosure.

The kits provided herein generally include instructions for carrying out the methods detailed below. Instructions included in the kits may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips, storage drives), optical media (e.g., CD ROM), and the like. As used herein, the term “instructions” can include the address of an internet site that provides the instructions. The kit can also include instructions in one or more languages, for example in more than one language.

In one aspect, the disclosure provides kits containing any one or more of the elements disclosed in the above methods and compositions. In one particular embodiment, the kit comprises a solution including a polyanionic polymer or salt thereof, or combinations of polyanionic polymers or salts thereof, and one or more buffers. In another particular embodiment, the kit comprises one or more of a Cas protein (for example, a recombination Cas protein, such as Cas9 or Cas12, or another Cas protein described above), a solution containing polyanionic polymer or salt thereof (for example, dextran sulfate sodium salt and/or another sulfated polysaccharide described above), a reconstitution solution, and a dilution solution. Suitable and exemplary reconstitution solutions are those including, for example, 50% glycerol. Suitable and exemplary dilution solutions are those including, for example, 20 mM HEPES buffer (pH 7.5) and 20 mM NaCl. One exemplary polyanionic polymer solution includes 1 μg/μL dextran sulfate sodium salt in 2 mM HEPES buffer (pH 7.5) and 20 mM NaCl.

### Definitions

The following definitions and methods are provided to better define the present disclosure and to guide those of ordinary skill in the art in the practice of the present disclosure. Unless otherwise noted, terms are to be understood according to conventional usage by those of ordinary skill in the relevant art.

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd Ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “about” when used in relation to a numerical value, x, for example means x±5%.

As used herein, the terms “complementary” or “complementarity” refer to the association of double-stranded nucleic acids by base pairing through specific hydrogen bonds. The base paring may be standard Watson-Crick base pairing (e.g., 5′-A G T C-3′ pairs with the complementary sequence 3′-T C A G-5′). The base pairing also may be Hoogsteen or reversed Hoogsteen hydrogen bonding. Complementarity is typically measured with respect to a duplex region and thus, excludes overhangs, for example. Complementarity between two strands of the duplex region may be partial and expressed as a percentage (e.g., 70%), if only some (e.g., 70%) of the bases are complementary. The bases that are not complementary are “mismatched.” Complementarity may also be complete (i.e., 100%), if all the bases in the duplex region are complementary.

As used herein, the term “CRISPR system” refers to a complex comprising a CRISPR protein (i.e., nuclease, nickase, or catalytically dead protein) and a guide RNA.

The term “endogenous” or “endogenous sequence,” as used herein, refers to a chromosomal sequence that is native to the cell.

The terms “target sequence,” “target chromosomal sequence,” and “target site” are used interchangeably to refer to the specific sequence in chromosomal DNA to which the programmable DNA modification protein is targeted, and the site at which the programmable DNA modification protein modifies the DNA or protein(s) associated with the DNA.

As used herein, the term “exogenous” or “exogenous sequence” refers to a sequence that is not native to the cell, or a chromosomal sequence whose native location in the genome of the cell is in a different chromosomal location.

A “gene,” as used herein, refers to a DNA region (including exons and introns) encoding a gene product, as well as all DNA regions which regulate the production of the gene product, whether or not such regulatory sequences are adjacent to coding and/or transcribed sequences.

Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The term “heterologous” refers to an entity that is not endogenous or native to the cell of interest. For example, a heterologous protein refers to a protein that is derived from or was originally derived from an exogenous source, such as an exogenously introduced nucleic acid sequence. In some instances, the heterologous protein is not normally produced by the cell of interest.

The term “nickase” refers to an enzyme that cleaves one strand of a double-stranded nucleic acid sequence (i.e., nicks a double-stranded sequence). For example, a nuclease with double strand cleavage activity can be modified by mutation and/or deletion to function as a nickase and cleave only one strand of a double-stranded sequence.

The term “nuclease,” as used herein, refers to an enzyme that cleaves both strands of a double-stranded nucleic acid sequence.

The terms “nucleic acid” and “polynucleotide” refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

The term “nucleotide” refers to deoxyribonucleotides or ribonucleotides. The nucleotides may be standard nucleotides (i.e., adenosine, guanosine, cytidine, thymidine, and uridine), nucleotide isomers, or nucleotide analogs. A nucleotide analog refers to a nucleotide having a modified purine or pyrimidine base or a modified ribose moiety. A nucleotide analog may be a naturally occurring nucleotide (e.g., inosine, pseudouridine, etc.) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos.

The terms “polypeptide” and “protein” are used interchangeably to refer to a polymer of amino acid residues.

As used herein, the term “programmable DNA modification protein” refers to a protein that is engineered to bind a specific target sequence in chromosomal DNA and which modifies the DNA or protein(s) associated with DNA at or near the target sequence.

The term “sequence identity” as used herein, indicates a quantitative measure of the degree of identity between two sequences of substantially equal length. The percent identity of two sequences, whether nucleic acid or amino acid sequences, is the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14 (6): 6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website. In general, the substitutions are conservative amino acid substitutions: limited to exchanges within members of group 1: glycine, alanine, valine, leucine, and Isoleucine; group 2: serine, cysteine, threonine, and methionine; group 3: proline; group 4: phenylalanine, tyrosine, and tryptophan; group 5: aspartate, glutamate, asparagine, and glutamine.

The terms “target sequence,” “target chromosomal sequence,” and “target site” are used interchangeably to refer to the specific sequence in chromosomal DNA to which the programmable DNA modification protein is targeted, and the site at which the programmable DNA modification protein modifies the DNA or protein(s) associated with the DNA.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion.

In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity.

As various changes could be made in the above-described methods and compositions without departing from the scope of the disclosure, it is intended that all matter contained in the above description and in the examples given below, shall be interpreted as illustrative and not in a limiting sense.

Having described the inventions in detail, it will be apparent that modifications and variations are possible without departing the scope of the inventions defined in the appended claims. Furthermore, it should be appreciated that all examples in the present disclosure are provided as non-limiting examples.

### EXAMPLES

The following non-limiting examples are provided to further illustrate the present inventions. It should be appreciated by those of skill in the art that the techniques disclosed in the examples that follow represent approaches the inventors have found function well in the practice of the inventions, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the inventions.

### Example 1: Dextran Sulfate Dosage-Dependent Enhancement on SpCas9 Endonuclease Genome Modification Efficiency in Human Cells

SpCas9 protein (Product number: CAS9PROT) and a synthetic single guide RNA (sgRNA) with the spacer sequence of 5′-GAGGGGGAACAGUUCUGAAA-3′ (SEQ ID NO: 33) targeting the human RelA locus were purchased from MilliporeSigma.

TABLE 1
RelA Target Site and PAM

| Category | Sequence (5′-3′) |
|---|---|
| RelA target site | 5′-GAGGGGGAACAGTTCTGAAAGGG- |
| protospacer and | 3′ (SEQ ID NO: 34) |
| PAM (underlined): | |

Dextran sulfate sodium salt with average molecular weight greater than 500 kDa (Product number: D8906) was also purchased from MilliporeSigma. A dextran sulfate solution was prepared by dissolving the chemical in water at 50 μg/μL and sterilized by filtration through a 0.22 μm filter. The stock solution was diluted with water to prepare working solutions.

Ribonucleoprotein (RNP) complexes were prepared by adding a buffer (20 mM HEPES, 100 mM KCl, 0.5 mM DTT, 0.1 mM EDTA, pH 7.5), 100 pmol sgRNA, and 5 μg of Cas9 protein to a 1.5-mL microcentrifuge tube in a 10 μL total reaction volume. The sgRNA to Cas9 protein molar ratio is approximately 3:1. The complexes were incubated at room temperature for 15 minutes and then kept on ice until transfection. Human K562 cells were seeded at 0.25×10⁶ cells per mL one day prior to transfection and were at approximately 0.5×10⁶ cells per mL at the time of transfection. Cells were washed twice with Hank's Balanced Salt Solution and then resuspended in Nucleofector Solution V (Lonza) at approximately 0.35×10⁶ cells per 100 μL. Nucleofection was performed by first mixing 100 μL of cells with 1 μL of dextran sulfate solution and then mixing with RNP complexes by gently pipetting up and down without introducing air bubbles before transferring into a cuvette for electroporation with Amaxa program T-016. Cells were immediately transferred to a 6-well plate containing 2 mL pre-warmed medium per well and grown at 37° C. and 5% CO₂ for 3 days before being harvested for genomic modification assays.

Genomic DNA extracts from transfected cells were prepared using QuickExtract Solution. Targeted genomic region was PCR amplified with NGS primers using JumpStart™ Taq ReadyMix™ for Quantitative PCR Kit (MilliporeSigma) with the following cycling condition: 98° C./2 m; 98° C./15 s, 62° C./30 s, and 72° C./45 s for 34 cycles; 72° C./5 m.

TABLE 2
NGS Primers for RelA Target Site

5′-
TCGTCGGCAGCGTCAGATGTGTATAAGAGACAGNNNNNNCTGCTTGG
GCTGCTCAATGA-3′ (SEQ ID NO: 35) (forward)
5′-
GTCTCGTGGGCTCGGAGATGTGTATAAGAGACAGNNNNNNGTTCCTTT
CCTCCACCCCTT-3′ (SEQ ID NO: 36) (reverse)

Primary PCR products were then reamplified with Illumina index primers using JumpStart™ Taq ReadyMix™ for Quantitative PCR Kit (MilliporeSigma) with the following cycling condition: 95° C./3 m; 95° C./30 s, 55° C./30 s, and 72° C./30 s for 8 cycles; 72° C./5 m. Indexed PCR products were purified with Select-a-Size DNA Clean & Concentrator kit (Zymo) and quantified by PicoGreen (ThermoFisher). PCR products were then normalized and pooled to make NGS libraries. NGS was performed using an Illumina MiSeq instrument and a 2×300 bp kit. The FASTQ files for each sample were analyzed for genome editing frequency.

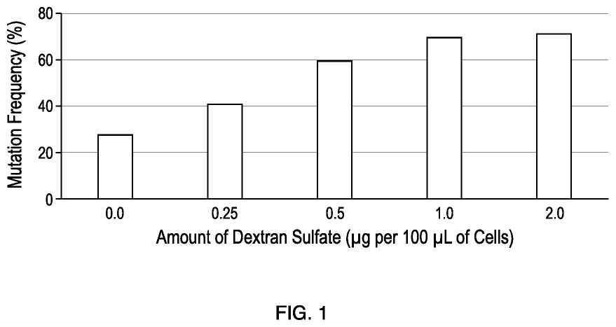

Results are presented in FIG. 1. The results show that the nuclease activity was enhanced when cells were treated with a dextran sulfate solution and the magnitude of enhancement increased as the dosage of dextran sulfate increased from 0.25 μg to about 1 μg per 100 μL of cells.

### Example 2: Dextran Sulfate Enhances SpCas9 Nuclease Editing Efficiency at Different Endogenous Targets in Human Cells

SpCas9 protein (Product number: CAS9PROT) and synthetic sgRNAs targeting six human genomic sites were purchased from MilliporeSigma. Each genomic site was tested in three biological replicates. The spacer sequences of these sgRNAs are listed in Table 1. Dextran sulfate sodium salt solution was prepared as described in Example 1 and diluted with water to prepare a working solution at 1 μg/μL. RNP complexes were prepared using 5 μg of SpCas9 protein and 100 pmol sgRNA as described in Example 1. Human k562 cells were seeded at 0.25×10⁶ cells per mL one day prior to transfection and were at approximately 0.5×10⁶ cells per mL at the time of transfection. Cells were washed twice with Hank's Balanced Salt Solution and then resuspended at approximately 0.35×10⁶ cells per 100 μL in Nucleofector Solution V supplemented with 1 μL of dextran sulfate at 1 μg/μL or 1 μL of water (control) per 100 μL. Nucleofection was performed by transferring 100 μL of cells into RNP complexes and mixing immediately by gently pipetting up and down without introducing air bubbles before transferring into a cuvette for electroporation with Amaxa program T-016. Cells were immediately transferred to a 6-well plate containing 2 mL pre-warmed medium per well and grown at 37° C. and 5% CO₂ for 3 days before being harvested for genomic modification assays.

Genomic DNA extracts from transfected cells were prepared using QuickExtract Solution. For CAR19, CCR5, CHI3L1, POR23, and VEGFA, targeted genomic regions were PCR amplified with NGS primers using JumpStart™ Taq ReadyMix™ for Quantitative PCR Kit (MilliporeSigma) with the following cycling condition: 98° C./2 m; 98° C./15 s, 62° C./30 s, and 72° C./45 s for 34 cycles; 72° C./5 m. For HEKSite4, targeted genomic region was PCR amplified with NGS primers using KAPA HiFi HotStart ReadyMix PCR Kit (Roche) with the following cycling condition: 95° C./3 m; 98° C./20 s, 68° C./30 s, and 72° C./45 s for 34 cycles; 72° C./5 m. Target sites and PAMs are listed in Table 3 and the NGS primers are listed in Table 4. Primary PCR products were then reamplified with Illumina index primers using JumpStart™ Taq ReadyMix™ for Quantitative PCR Kit (MilliporeSigma) with the following cycling condition: 95° C./3 m; 95° C./30 s, 55° C./30 s, and 72° C./30 s for 8 cycles; 72° C./5 m. Indexed PCR products were purified with Select-a-Size DNA Clean & Concentrator kit (Zymo) and quantified by PicoGreen (ThermoFisher). PCR products were then normalized and pooled to make NGS libraries. NGS was performed using an Illumina MiSeq instrument and a 2×300 bp kit. The FASTQ files for each sample were analyzed for genome editing frequency.

TABLE 3
Target Sites and PAMs

| Category | Sequence (5′-3′) |
|---|---|
| CAR19 target site protospacer and | 5′- |
| PAM (underlined): | CTTTAATGCGCTGACTTGTGAGG-3′ |
| | (SEQ ID NO: 37) |
| CCR5 target site protospacer and | 5′-TGACATCAATTATTATACATCGG- |
| PAM (underlined): | 3′ (SEQ ID NO: 38) |
| CHI3L1 target site protospacer | 5′- |
| and PAM (underlined): | AGCTGGTCCCAGTACCGGGAAGG- |
| | 3′ (SEQ ID NO: 39) |
| HEKSite4 target site protospacer | 5′- |
| and PAM (underlined): | GGCACTGCGGCTGGAGGTGGGGG- |
| | 3′ (SEQ ID NO: 40) |
| POR23 target site protospacer and | 5′- |
| PAM (underlined): | AGCCGTGAGTGGAGGGAGCGTGG- |
| | 3′ (SEQ ID NO: 41) |
| VEGFA target site protospacer | 5′- |
| and PAM (underlined): | GGGTGGGGGGAGTTTGCTCCTGG- |
| | 3′ (SEQ ID NO: 42) |

TABLE 4
sgRNA spacer and NGS primer sequences

| Category | Sequence (5′-3′) |
|---|---|
| CAR19 | CUUUAAUGCGCUGACUUGUG (SEQ ID NO: 43) |
| sgRNA | |
| spacer | |
| CCR5 | UGACAUCAAUUAUUAUACAU (SEQ ID NO: 44) |
| sgRNA | |
| spacer | |
| CHI3L1 | AGCUGGUCCCAGUACCGGGA (SEQ ID NO: 45) |
| sgRNA | |
| spacer | |
| HEKSite4 | GGCACUGCGGCUGGAGGUGG (SEQ ID NO: 46) |
| sgRNA | |
| spacer | |
| POR23 | AGCCGUGAGUGGAGGGAGCG (SEQ ID NO: 47) |
| sgRNA | |
| spacer | |
| VEGFA | GGGUGGGGGGAGUUUGCUCC (SEQ ID NO: 48) |
| sgRNA | |
| spacer | |
| CAR19 | TCGTCGGCAGCGTCAGATGTGTATAAGAGACAGNNNNNNCT |
| NGS | GACTGCTGACCCCTCCTA (SEQ ID NO: 49) |
| forward | |
| primer | |
| CAR19 | GTCTCGTGGGCTCGGAGATGTGTATAAGAGACAGNNNNNNG |
| NGS | CCACAGGGTAGTTAGACTCTAAG (SEQ ID NO: 50) |
| reverse | |
| primer | |
| CCR5 | TCGTCGGCAGCGTCAGATGTGTATAAGAGACAGNNNNNNCA |
| NGS | TGGAGGGCAACTAAATACATTCTAGGAC (SEQ ID NO: 51) |
| forward | |
| primer | |
| CCR5 | GTCTCGTGGGCTCGGAGATGTGTATAAGAGACAGNNNNNNT |
| NGS | CAGAGATGGCCAGGTTGAGC (SEQ ID NO: 52) |
| reverse | |
| primer | |
| CHI3L1 | TCGTCGGCAGCGTCAGATGTGTATAAGAGACAGNNNNNNGT |
| NGS | TCTTGAGTGTGTTGAGCATGCC (SEQ ID NO: 53) |
| forward | |
| primer | |
| CHI3L1 | GTCTCGTGGGCTCGGAGATGTGTATAAGAGACAGNNNNNNT |
| NGS | GCTCACAATCTGCACCCCAC (SEQ ID NO: 54) |
| reverse | |
| primer | |
| HEKSite4 | TCGTCGGCAGCGTCAGATGTGTATAAGAGACAGNNNNNNGG |
| NGS | AACCCAGGTAGCCAGAGA (SEQ ID NO: 55) |
| forward | |
| primer | |
| HEKSite4 | GTCTCGTGGGCTCGGAGATGTGTATAAGAGACAGNNNNNNG |
| NGS | GGGTGGGGTCAGACGT (SEQ ID NO: 56) |
| reverse | |
| primer | |
| POR23 | TCGTCGGCAGCGTCAGATGTGTATAAGAGACAGNNNNNNTG |
| NGS | GTCTCCCCTTTCCAGCATTC (SEQ ID NO: 57) |
| forward | |
| primer | |
| POR23 | GTCTCGTGGGCTCGGAGATGTGTATAAGAGACAGNNNNNNA |
| NGS | ACTGGGCGAGTGCCAGTCT (SEQ ID NO: 58) |
| reverse | |
| primer | |
| VEGFA | TCGTCGGCAGCGTCAGATGTGTATAAGAGACAGNNNNNNGA |
| NGS | CACACACGTCCTCACTCTC (SEQ ID NO: 59) |
| forward | |
| primer | |
| VEGFA | GTCTCGTGGGCTCGGAGATGTGTATAAGAGACAGNNNNNNT |
| NGS | GTCTGCAGGCCAGATGAGG (SEQ ID NO: 60) |
| reverse | |
| primer | |

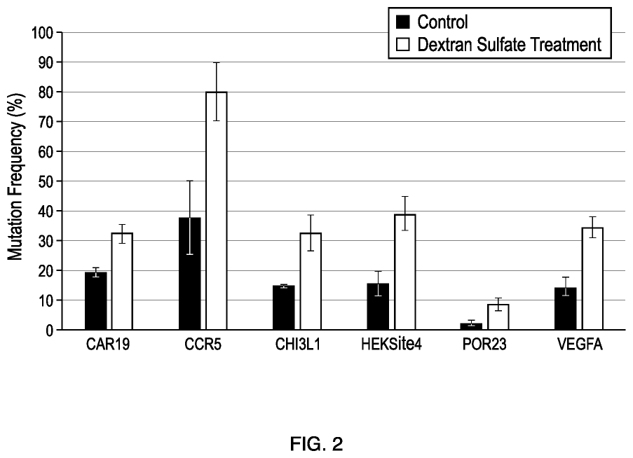

Results are presented in FIG. 2. The results show that dextran sulfate treatment significantly increased the editing efficiency of SpCas9 at every target site tested. Compared with the control, the magnitude of improvement by the enhancer ranged between approximately two to threefold, dependent of the target site.

### Example 3. Dextran Sulfate Enhances the C to T Editing Efficiency of a Cytosine Base Editor at Different Endogenous Sites

A cytosine base editor was constructed by fusing a human APOBEC3A to the amino acid terminus of a SpCas9 D10A nickase. The recombinant protein of the base editor was purified from E. coli to over 90% homogeneity. Synthetic sgRNAs targeting 6 endogenous sites in human cells were purchased from MilliporeSigma. Each site was tested in three biological replicates. The spacer sequences of these sgRNAs are listed in Table 5. Dextran sulfate sodium salt solution was prepared as described in Example 1 and diluted with water to prepare a working solution at 1 μg/μL. Ribonucleoprotein (RNP) complexes were prepared by adding a buffer (20 mM HEPES, 100 mM KCl, 0.5 mM DTT, 0.1 mM EDTA, pH 7.5), 200 pmol sgRNA, and 15 μg of the cytosine base editor protein to a 1.5-mL microcentrifuge tube in a 10 μL total reaction volume. The sgRNA to cytosine base editor protein molar ratio is approximately 3:1. The complexes were incubated at room temperature for 15 minutes and then kept on ice until transfection. Human HEK293 cells were seeded at approximately 20% confluency two days prior to transfection and were at approximately 80% confluency at the time of transfection. Cells were detached with a trypsin solution and washed twice with Hank's Balanced Salt Solution and then resuspended at approximately 0.3×10⁶ cells per 100 μL in Nucleofector Solution V supplemented with 1 μL of dextran sulfate at 1 μg/μL or 1 μL of water (control) per 100 μL. Nucleofection was performed by transferring 100 μL of cells into RNP complexes and mixing immediately by gently pipetting up and down without introducing air bubbles before transferring into a cuvette for electroporation with Amaxa program Q-001. Cells were immediately transferred to a 6-well plate containing 2 mL pre-warmed medium per well and grown at 37° C. and 5% CO₂ for 3 days before being harvested for genomic modification assays.

Genomic DNA extracts from transfected cells were prepared using QuickExtract Solution. Targeted genomic regions were PCR amplified with NGS primers using JumpStart™ Taq ReadyMix™ for Quantitative PCR Kit (MilliporeSigma) with the following cycling condition: 98° C./2m; 98° C./15s, 62° C./30s, and 72° C./45s for 34 cycles; 72° C./5m. The NGS primers are listed in Table 5. Primary PCR products were then reamplified with Illumina index primers using JumpStart™ Taq ReadyMix™ for Quantitative PCR Kit (MilliporeSigma) with the following cycling condition: 95° C./3m; 95° C./30s, 55° C./30s, and 72° C./30s for 8 cycles; 72° C./5m. Indexed PCR products were purified with Select-a-Size DNA Clean & Concentrator kit (Zymo) and quantified by PicoGreen (ThermoFisher). PCR products were then normalized and pooled to make NGS libraries. NGS was performed using an Illumina MiSeq instrument and a 2×300 bp kit. The FASTQ files for each sample were analyzed for C to T conversion frequency.

TABLE 5
Target Sites and PAMs

| Category | Sequence (5′-3′) |
|---|---|
| CCR5 target site protospacer and | 5′-TGACATCAATTATTATACATCGG- |
| PAM (underlined): | 3′ (SEQ ID NO: 38) |
| RNF2 target site protospacer and | 5′-GTCATCTTAGTCATTACCTGAGG- |
| PAM (underlined): | 3′ (SEQ ID NO: 76) |
| HEK293_site 2 target site | 5′- |
| protospacer and PAM | GAACACAAAGCATAGACTGCGGG- |
| (underlined): | 3′ (SEQ ID NO: 77) |
| HEK293_site 3 target site | 5′- |
| protospacer and PAM | GGCCCAGACTGAGCACGTGATGG- |
| (underlined): | 3′ (SEQ ID NO: 78) |
| EMX1 target site protospacer and | 5′- |
| PAM (underlined): | GAGTCCGAGCAGAAGAAGAAGG-3′ |
| | (SEQ ID NO: 79) |
| HBB target site protospacer and | 5′- |
| PAM (underlined): | CTTGCCCCACAGGGCAGTAACGG- |
| | 3′ (SEQ ID NO: 80) |

TABLE 6
sgRNA spacer and NGS primer sequences

| Category | Sequence (5′-3′) |
|---|---|
| CCR5 | UGACAUCAAUUAUUAUACAU (SEQ ID NO: 44) |
| sgRNA | |
| spacer | |
| HEK293_ | GAACACAAAGCAUAGACUGC (SEQ ID NO: 61) |
| site2 | |
| sgRNA | |
| spacer | |
| EMX1 | GAGUCCGAGCAGAAGAAGAA (SEQ ID NO: 62) |
| sgRNA | |
| spacer | |
| RNF2 | GUCAUCUUAGUCAUUACCUG (SEQ ID NO: 63) |
| sgRNA | |
| spacer | |
| HEK293_ | GGCCCAGACUGAGCACGUGA (SEQ ID NO: 64) |
| site3 | |
| sgRNA | |
| spacer | |
| HBB | CUUGCCCCACAGGGCAGUAA (SEQ ID NO: 65) |
| sgRNA | |
| spacer | |
| CCR5 | TCGTCGGCAGCGTCAGATGTGTATAAGAGACAGNNNNNNCA |
| NGS | TGGAGGGCAACTAAATACATTCTAGGAC (SEQ ID NO: 51) |
| forward | |
| primer | |
| CCR5 | GTCTCGTGGGCTCGGAGATGTGTATAAGAGACAGNNNNNNT |
| NGS | CAGAGATGGCCAGGTTGAGC (SEQ ID NO: 52) |
| reverse | |
| primer | |
| HEK293_ | TCGTCGGCAGCGTCAGATGTGTATAAGAGACAGNNNNNNTG |
| site2 NGS | AACTTCCCAAGTGAGAAGCCAG (SEQ ID NO: 66) |
| forward | |
| primer | |
| HEK293_ | GTCTCGTGGGCTCGGAGATGTGTATAAGAGACAGNNNNNNT |
| site2 NGS | GGCAGGACGTCTGCCCAAT (SEQ ID NO: 67) |
| reverse | |
| primer | |
| EMX1 | TCGTCGGCAGCGTCAGATGTGTATAAGAGACAGNNNNNNCC |
| NGS | CCAGTGGCTGCTCT (SEQ ID NO: 68) |
| forward | |
| primer | |
| EMX1 | GTCTCGTGGGCTCGGAGATGTGTATAAGAGACAGNNNNNNC |
| NGS | CAGGCCTCCCCAAAGC (SEQ ID NO: 69) |
| reverse | |
| primer | |
| RNF2 | TCGTCGGCAGCGTCAGATGTGTATAAGAGACAGNNNNNNTG |
| NGS | TTAGCCAACATACAGAAGTCAGGAATGC (SEQ ID NO: 70) |
| forward | |
| primer | |
| RNF2 | GTCTCGTGGGCTCGGAGATGTGTATAAGAGACAGNNNNNNT |
| NGS | TCAGACCATAGCACTTCCCTTCC (SEQ ID NO: 71) |
| reverse | |
| primer | |
| HEK293 | TCGTCGGCAGCGTCAGATGTGTATAAGAGACAGNNNNNNAC |
| site3 NGS | CTCCCTAGGTGCTGGCTT (SEQ ID NO: 72) |
| forward | |
| primer | |
| HEK293 | GTCTCGTGGGCTCGGAGATGTGTATAAGAGACAGNNNNNNG |
| site3 NGS | CTGATGTGGGCTGCCTAGA (SEQ ID NO: 73) |
| reverse | |
| primer | |
| HBB NGS | TCGTCGGCAGCGTCAGATGTGTATAAGAGACAGNNNNNNGC |
| forward | CAATCTACTCCCAGGAGC (SEQ ID NO: 74) |
| primer | |
| HBB NGS | GTCTCGTGGGCTCGGAGATGTGTATAAGAGACAGNNNNNNA |
| reverse | GGCAGAGAGAGTCAGTGCCTA (SEQ ID NO: 75) |
| primer | |

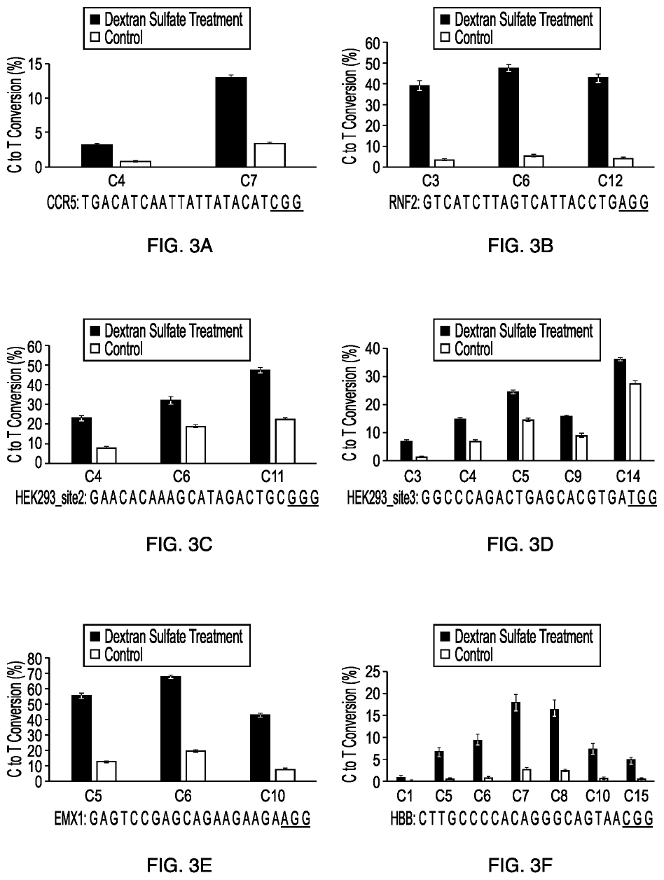

Results are presented in FIGS. 3A-3F. The results show that dextran sulfate treatment increased the C to T conversion efficiency at every C within the editing window of the cytosine base editor and at every target site tested as compared with the control. The improvement ranged from approximately twofold to tenfold, dependent on the position of the converted C on the protospacer and the target site.

### Example 4. Pentosan Polysulfate Enhancement on Cas9 Nuclease Activity

A high fidelity SpCas9 protein (Catalog No. CAS9 Plus) and a synthetic single guide RNA (sgRNA) with the spacer sequence of 5′-GGCACUGCGGCUGGAGGUGG-3′ (SEQ ID NO: 46) targeting the human HEKSite4 locus were purchased from MilliporeSigma. Pentosan polysulfate sodium salt with a formula of (C₅H₆Na₂O₁₀S₂)ₙ was purchased from Selleckchem (Catalog No. S3500). A pentosan polysulfate solution was prepared by dissolving the chemical in water at 50 mg/ml. The molecular structural unit of the polyanionic polymer is as follows:

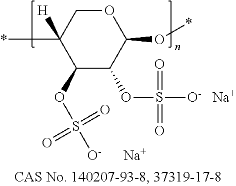

Ribonucleoprotein (RNP) complexes were prepared by adding a buffer (20 mM HEPES, 100 mM KCl, 0.5 mM DTT, 0.1 mM EDTA, pH 7.5), 100 pmol sgRNA, and 5 μg of Cas9 protein to a 1.5-mL microcentrifuge tube in a 10 μL total reaction volume. The sgRNA to Cas9 protein molar ratio is approximately 3:1. The complexes were incubated at room temperature for 15 minutes and then kept on ice until transfection. Human dermal fibroblast cells were purchased from MilliporeSigma (Product number: 106-05A) and seeded at approximately 20% confluency two days prior to transfection and were at approximately 80% confluency at the time of transfection. Human HEK293 cells were purchased from ATCC (Catalog No. CRL 1573) and seeded at approximately 20% confluency two days prior to transfection and were at approximately 80% confluency at the time of transfection. Cells were detached by trypsinization and washed twice with Hank's Balanced Salt Solution and then resuspended in Nucleofector Solution VPD-1001 (Lonza) for fibroblasts or Nucleofector Solution V (Lonza) for HEK293 at approximately 0.25×10⁶ cells per 100 μL. Both Nucleofector Solutions were supplemented with pentosan polysulfate solution at different dosages before cell resuspension. Nucleofection was performed by mixing 100 μL of cells with RNP complexes by gently pipetting up and down without introducing air bubbles before transferring into a cuvette for electroporation. Fibroblasts and HEK293 were transfected with Amaxa programs U-023 and T-016, respectively. Cells were immediately transferred to a 6-well plate containing 2 mL pre-warmed medium per well and grown at 37° C. and 5% CO₂ for 3 days before being harvested for genomic modification assays. Fibroblast transfection was performed in duplicate.

Genomic DNA extracts from transfected cells were prepared using QuickExtract Solution. Targeted genomic region was PCR amplified with NGS primers using JumpStart™ Taq ReadyMix™ for Quantitative PCR Kit (MilliporeSigma) with the following cycling condition: 98° C./2 m; 98° C./15 s, 62° C./30 s, and 72° C./45 s for 34 cycles; 72° C./5 m.

TABLE 7
NGS Primers for HEKSite4 Target Site

5′-
TCGTCGGCAGCGTCAGATGTGTATAAGAGACAGNNNNNNGGAACCCA
GGTAGCCAGAGA-3′ (SEQ ID NO: 55) (forward)
5′-
GTCTCGTGGGCTCGGAGATGTGTATAAGAGACAGNNNNNNGGGGTGG
GGTCAGACGT-3′ (SEQ ID NO: 56) (reverse)

Primary PCR products were then reamplified with Illumina index primers using JumpStart™ Taq ReadyMix™ for Quantitative PCR Kit (MilliporeSigma) with the following cycling condition: 95° C./3 m; 95° C./30 s, 55° C./30 s, and 72° C./30 s for 8 cycles; 72° C./5 m. Indexed PCR products were purified with Select-a-Size DNA Clean & Concentrator kit (Zymo) and quantified by PicoGreen (ThermoFisher). PCR products were then normalized and pooled to make NGS libraries. NGS was performed using an Illumina MiSeq instrument and a 2×300 bp kit. The FASTQ files for each sample were analyzed for genome editing frequency.

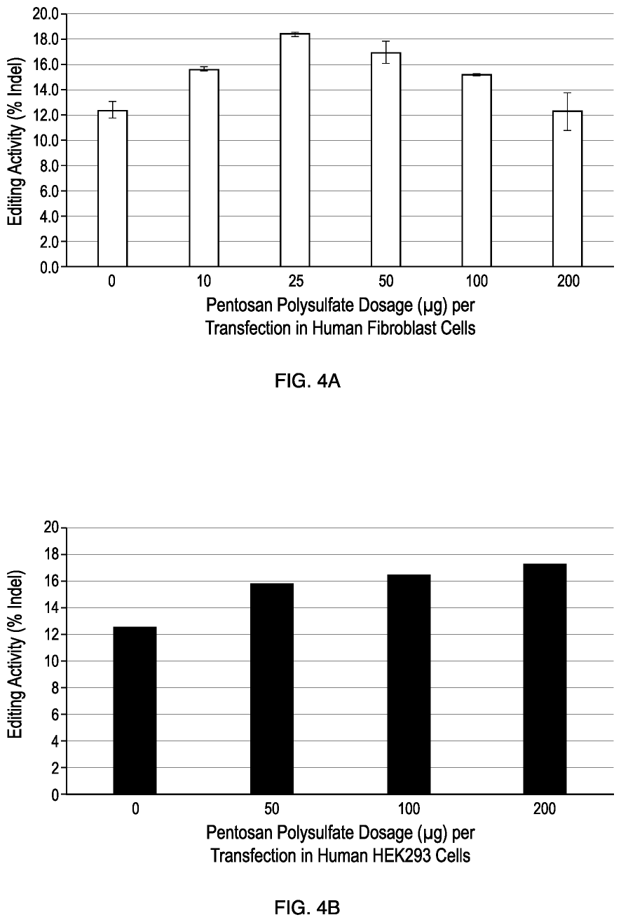

Results are presented in FIG. 4A and FIG. 4B. The results show that Cas9 nuclease activity was enhanced when cells were treated with an appropriate dosage of pentosan polysulfate.

### Example 5. Heparan Sulfate Enhancement on Cas9 Nuclease Activity

A high fidelity SpCas9 protein (Catalog No. CAS9 Plus) and a synthetic single guide RNA (sgRNA) with the spacer sequence of 5′-GGCACUGCGGCUGGAGGUGG-3′ (SEQ ID NO: 46) targeting the human HEKSite4 locus were purchased from MilliporeSigma. Heparan sulfate was purchased from Selleckchem (Catalog No. S5992). A heparan sulfate solution was prepared by dissolving the chemical in water at 50 mg/ml. The molecular structural unit of the polyanionic polymer is as follows:

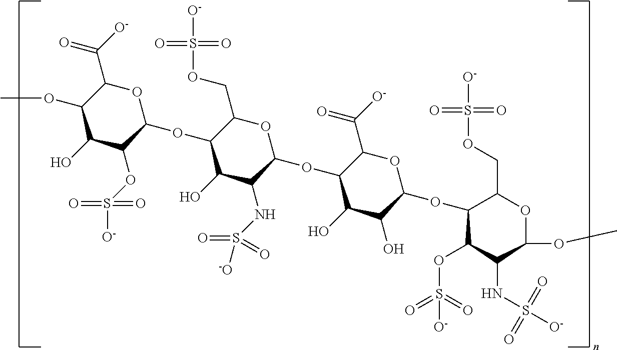

Ribonucleoprotein (RNP) complexes were prepared by adding a buffer (20 mM HEPES, 100 mM KCl, 0.5 mM DTT, 0.1 mM EDTA, pH 7.5), 100 pmol sgRNA, and 5 μg of Cas9 protein to a 1.5-mL microcentrifuge tube in a 10 μL total reaction volume. The sgRNA to Cas9 protein molar ratio is approximately 3:1. The complexes were incubated at room temperature for 15 minutes and then kept on ice until transfection. Human dermal fibroblast cells were purchased from MilliporeSigma (Product number: 106-05A) and seeded at approximately 20% confluency two days prior to transfection and were at approximately 80% confluency at the time of transfection. Human HEK293 cells were purchased from ATCC (Catalog No. CRL 1573) and seeded at approximately 20% confluency two days prior to transfection and were at approximately 80% confluency at the time of transfection. Cells were detached by trypsinization and washed twice with Hank's Balanced Salt Solution and then resuspended in Nucleofector Solution VPD-1001 (Lonza) for fibroblasts or Nucleofector Solution V (Lonza) for HEK293 at approximately 0.25×10⁶ cells per 100 μL. Both Nucleofector Solutions were supplemented with heparan sulfate solution at different dosages before cell resuspension. Nucleofection was performed by mixing 100 μL of cells with RNP complexes by gently pipetting up and down without introducing air bubbles before transferring into a cuvette for electroporation. Fibroblasts and HEK293 were transfected with Amaxa programs U-023 and T-016, respectively. Cells were immediately transferred to a 6-well plate containing 2 mL pre-warmed medium per well and grown at 37° C. and 5% CO₂ for 3 days before being harvested for genomic modification assays. Fibroblast transfection was performed in duplicate.

Genomic DNA extracts from transfected cells were prepared using QuickExtract Solution. Targeted genomic region was PCR amplified with NGS primers using JumpStart™ Taq ReadyMix™ for Quantitative PCR Kit (MilliporeSigma) with the following cycling condition: 98° C./2 m; 98° C./15 s, 62° C./30 s, and 72° C./45 s for 34 cycles; 72° C./5 m.

TABLE 8
NGS Primers for HEKSite4 Target Site

5′-
TCGTCGGCAGCGTCAGATGTGTATAAGAGACAGNNNNNNGGAACCCA
GGTAGCCAGAGA-3′ (SEQ ID NO: 55) (forward)
5′-
GTCTCGTGGGCTCGGAGATGTGTATAAGAGACAGNNNNNNGGGGTGG
GGTCAGACGT-3′ (SEQ ID NO: 56) (reverse)

Primary PCR products were then reamplified with Illumina index primers using JumpStart™ Taq ReadyMix™ for Quantitative PCR Kit (MilliporeSigma) with the following cycling condition: 95° C./3 m; 95° C./30 s, 55° C./30 s, and 72° C./30 s for 8 cycles; 72° C./5 m. Indexed PCR products were purified with Select-a-Size DNA Clean & Concentrator kit (Zymo) and quantified by PicoGreen (ThermoFisher). PCR products were then normalized and pooled to make NGS libraries. NGS was performed using an Illumina MiSeq instrument and a 2×300 bp kit. The FASTQ files for each sample were analyzed for genome editing frequency.

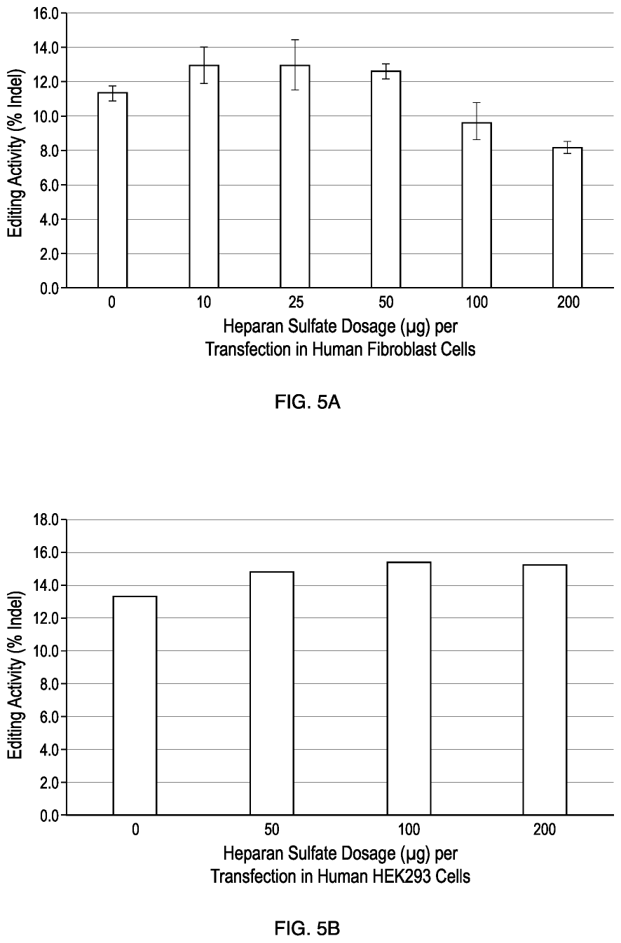

Results are presented in FIG. 5A and FIG. 5B. The results show that Cas9 nuclease activity was enhanced when cells were treated with an appropriate dosage of heparan sulfate.

### Example 6. Evaluation of Additional Compounds for Cas9 RNP Editing Enhancement by Cell Transfection

All compounds were purchased from MilliporeSigma and dissolved in a control buffer (2 mM HEPES, pH 7.5, 20 mM NaCl). Each compound was tested in three dosages as described in Table 1. A high fidelity SpCas9 protein (Catalog No. CAS9 Plus) and a synthetic single guide RNA (sgRNA) with the spacer sequence of 5′-GGCACUGCGGCUGGAGGUGG-3′ (SEQ ID NO: 46) targeting the human HEKSite4 locus were purchased from MilliporeSigma. Ribonucleoprotein (RNP) complexes were prepared by adding a buffer (20 mM HEPES, pH 7.5, 20 mM NaCl), 100 pmol sgRNA, and 5 μg of Cas9 protein to a 1.5-mL microcentrifuge tube in a 10 μL total reaction volume. The sgRNA to Cas9 protein molar ratio is approximately 3:1. The complexes were incubated at room temperature for 15 minutes and then kept on ice until transfection.

Human HEK293 cells were purchased from ATCC (Catalog No. CRL 1573) and seeded at approximately 20% confluency two days prior to transfection and were at approximately 80% confluency at the time of transfection. Cells were detached by trypsinization and washed twice with Hank's Balanced Salt Solution and then resuspended at approximately 0.25×10⁶ cells per 100 μL of Nucleofector Solution V (Lonza) supplemented with 2 μL of each compound. Each compound was combined with Nucleofector Solution V prior to cell resuspension. The control buffer (2 mM HEPES, pH 7.5, 20 mM NaCl) was also used at 2 μL per transfection. Nucleofection was performed by mixing 100 μL of cells with RNP complexes by gently pipetting up and down without introducing air bubbles before transferring into a cuvette for electroporation. Cells were transfected with Amaxa program T-016. Cells were immediately transferred to a 6-well plate containing 2 mL pre-warmed medium per well and grown at 37° C. and 5% CO₂ for 3 days before being harvested for genomic modification assays. Each transfection test was performed in duplicate

Genomic DNA extracts from transfected cells were prepared using QuickExtract Solution. Targeted genomic region was PCR amplified with NGS primers using JumpStart™ Taq ReadyMix™ for Quantitative PCR Kit (MilliporeSigma) with the following cycling condition: 98° C./2 m; 98° C./15 s, 62° C./30 s, and 72° C./45 s for 34 cycles; 72° C./5 m.

TABLE 9
NGS Primers for HEKSite4 Target Site

5′-
TCGTCGGCAGCGTCAGATGTGTATAAGAGACAGNNNNNNGGAACCCA
GGTAGCCAGAGA-3′ (SEQ ID NO: 55) (forward)
5′-
GTCTCGTGGGCTCGGAGATGTGTATAAGAGACAGNNNNNNGGGGTGG
GGTCAGACGT-3′ (SEQ ID NO: 56) (reverse)

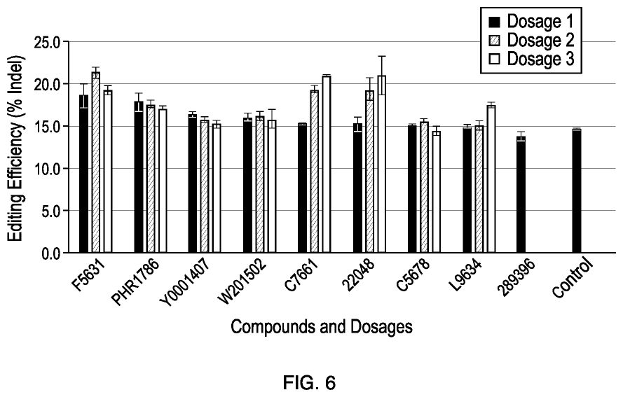

Primary PCR products were then reamplified with Illumina index primers using JumpStart™ Taq ReadyMix™ for Quantitative PCR Kit (MilliporeSigma) with the following cycling condition: 95° C./3 m; 95° C./30 s, 55° C./30 s, and 72° C./30 s for 8 cycles; 72° C./5 m. Indexed PCR products were purified with Select-a-Size DNA Clean & Concentrator kit (Zymo) and quantified by PicoGreen (ThermoFisher). PCR products were then normalized and pooled to make NGS libraries. NGS was performed using an Illumina MiSeq instrument and a 2×300 bp kit. The FASTQ files for each sample were analyzed for genome editing frequency. Results are presented in FIG. 6.

Results show that fucoidan from Fucus vesiculosus, chondroitin sulfate sodium, collagen from rat tail, K-carrageenan, and laminarin from laminaria digitata yielded a significantly higher level of editing efficiency than the control buffer with at least one of the three dosages tested.

TABLE 10
Compounds and dosages tested in cell
transfection

| | | Dosage 1 | Dosage 2 | Dosage 3 |
|---|---|---|---|---|
| Compound | Product | (μg per | (μg per | (μg per |
| Name | Number | transfection) | transfection) | transfection) |
| Fucoidan | F5631 | 1 | 10 | 100 |
| from Fucus | | | | |
| vesiculosus | | | | |
| Chondroitin | PHR1786 | 1 | 10 | 100 |
| sulfate sodium | | | | |
| Glucosamine | Y0001407 | 1 | 10 | 100 |
| sulfate sodium | | | | |
| chloride | | | | |
| Sodium alginate | W201502 | 1 | 10 | 20 |
| Collagen from rat | C7661 | 0.01 | 0.1 | 1 |
| tail | | | | |
| κ-Carrageenan | 22048 | 0.2 | 2 | 5 |
| Carboxymethylc | C5678 | 1 | 10 | 20 |
| ellulose sodium | | | | |
| salt | | | | |
| Laminarin | L9634 | 1 | 10 | 100 |
| from Laminaria | | | | |
| digitata | | | | |
| Hydroxyapatite | 289396 | <1 (nearly | | |
| | | insoluble) | | |

### Example 7. Dextran Sulfate Enhancement on Cas9 Nuclease Activity in Human Primary T-Cells

Human primary T-cell culture. CD8+ human primary T cells were purchased from StemCell. Cells were maintained in RPMI (Thermo) supplemented with 10% human AB serum (Sigma-Aldrich), 1× GlutaMAX™ (Gibco), 8 ng/ml IL-2 (Gibco), and 50 UM B-mercaptoethanol (Sigma). Cells were stimulated with Dynabeads™ Human T-Expander CD3/CD28 (Gibco) 3 days prior to nucleofection. Cells were cultured in the presence of Dynabeads™ post nucleofection according to manufacturer's protocol.

Cas9 RNP assembly and nucleofection. Dextran sulfate sodium salt with average molecular weight greater than 500 kDa (Product number: D8906) was purchased from MilliporeSigma. A dextran sulfate solution was prepared by dissolving the chemical in a control buffer (2 mM HEPES, pH 7.5, 20 mM NaCl) at 1 μg/μl and sterilized by filtration through a 0.22 μm filter. A high fidelity SpCas9 protein (Catalog No. CAS9 Plus) and a synthetic single guide RNA (sgRNA) with the spacer sequence of 5′-TCTGGTTGCTGGGGCTCATG-3′ (SEQ ID NO: 81) targeting the human PD-1 locus were also purchased from MilliporeSigma. Ribonucleoprotein (RNP) complexes were prepared by adding a buffer (20 mM HEPES, pH 7.5, 20 mM NaCl), 100 pmol sgRNA, and 5 μg of Cas9 protein to a 1.5-mL microcentrifuge tube in a 10 μL total reaction volume. The sgRNA to Cas9 protein molar ratio is approximately 3:1. The complexes were incubated at room temperature for 15 minutes and then kept on ice until transfection. In a 1.5 mL Eppendorf tube, 1,000,000 cells were resuspended with Lonza Nucleofector Solution and then the associated dose of dextran sulfate was added before the appropriate RNP complex was added yielding a total volume of 100 μL. Cells were transfected with Amaxa program DS-120 in a 4D-Nucleofector. Cells were immediately transferred to a 6-well plate containing 2 mL pre-warmed medium per well and grown at 37° C. and 5% CO₂ for 3 days before being harvested for genomic modification assays. Each transfection test was performed in duplicate.

PCR using tagged primers. Genomic DNA extracts from transfected cells were prepared using GenElute Mammalian Genomic DNA Kit (Sigma-Aldrich). JumpStart™ REDTaq® ReadyMix™ Reaction Mix (Sigma-Aldrich) along with primers flanking the genomic cut site of PD1 were used for PCR amplification. Primers were tagged with partial Illumina adapter sequences using the following primers:

TABLE 11
Primers

5′-
TCGTCGGCAGCGTCAGATGTGTATAAGAGACAGNNNNNNGGACAACG
CCACCTTCACCTGC-3′ (SEQ ID NO: 82) (forward)
5′-
GTCTCGTGGGCTCGGAGATGTGTATAAGAGACAGNNNNNNCTACGAC
CCTGGAGCTCCTGAT-3′ (SEQ ID NO: 83) (reverse)

The thermal cycling conditions included a heat denaturing step at 95° C. for 5 minutes followed by 34 cycles of 95° C. for 30s, anneal at 67.7° C. for 30s, and extension at 70° C. for 30s. Amplification was followed by a final extension at 70° C. for 10 min and a cool down to 4° C.

Sample indexing. A limited-cycle PCR was carried out to index the amplified PCR product. A total reaction volume of 50 μL included 25 μL JumpStart™ REDTaq® ReadyMix™ Reaction Mix (Sigma-Aldrich), 5 μL of amplified PCR product, 10 μL H₂O, and 5 μL each of 5 μM Nextera XT Index 1 (i7) and Index 2 (i5) oligos. The thermal cycling conditions consisted of an initial heat denature at 95° C. for 3 min, followed by 8 cycles of 95° C. for 30s, 55° C. for 30s, and 72° C. for 30s. A final extension was carried out at 72° C. for 5 min and the reaction was cooled down to 4° C. PCR purification was carried out using AxyPrep™ Mag PCR beads (Corning) and 25 μL of indexed sample at a 0.8:1 bead-to PCR ratio. DNA was eluted in 25 μL of 10 mM Tris.

Quantification of purified indexed PCR and library pooling. PicoGreen fluorescent dye (Invitrogen) was used for quantification of indexed samples. Purified indexed PCR was diluted to 1:100 with 1×TE. PicoGreen was diluted according to manufacturer's protocol (50 μL PicoGreen+10 mL 1×TE). Equal volume of diluted PicoGreen was added to the diluted indexed PCR sample yielding a final 1:1 dilution ratio in a fluorescence plate reader. Samples were excited at 475 nm and read at 530 nm. All samples were normalized to 4 nM with 1×TE, and 6 μL of each normalized sample was collected and pooled.

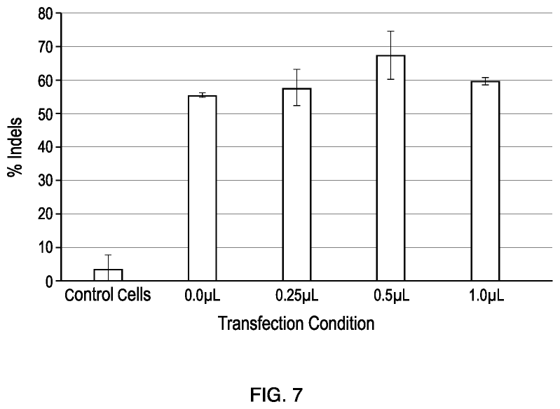

Library preparation for sequencing. Stock 10M NaOH was serial diluted with H₂O to yield a final 1:100 dilution (0.1M NaOH) the day of library preparation. To denature the DNA, 5 μL of 0.1M NaOH and 5 μL of the pooled 4 nM library were mixed together in an Eppendorf tube and incubated at RT for 5 minutes. 990 μL of cold Illumina HT1 buffer was added to the denatured DNA, yielding a 20 pM pooled, denatured library. Phix (20 pM) was thawed and 30 μL was transferred to a fresh tube. 570 μL of the 20 pM library was added to the PhiX resulting in 5% Phix for library diversification, quality control for cluster generation, sequencing, and alignment. This was mixed and heat shocked at 96° C. for 2 minutes and then immediately placed on ice. A 300 cycle v2 MiSeq reagent cartridge was thawed in an ice water bath and inverted to mix. A MiSeq v2 flow cell was rinsed thoroughly with water and ethanol and dried with a kimwipe making sure that all liquid was removed and no salt was detected on the surface of the cell. A p1200 tip was used to pierce the foil of well 17 of the thawed reagent cartridge. Using a new tip, 600 μL of the Phix containing library was added to well 17. Following the run, the FASTQ files for each sample were analyzed for genome editing frequency. Results are presented in FIG. 7.

Results show that dextran sulfate at the dosages of 0.5 and 1.0 μg per transfection yielded a significantly higher level of editing efficiency in human primary T-cells than the control buffer.

The invention claimed is:

1. A method for enhancing genome editing efficiency, the method comprising introducing, by electroporation, a programmable DNA modification system to a eukaryotic cell in the presence of a polyanionic polymer or salt thereof.

2. The method of claim 1, wherein genome editing efficiency is enhanced relative to an otherwise identical method in the absence of the polyanionic polymer or salt thereof.

3. (canceled)

4. (canceled)

5. (canceled)

6. The method of claim 1, wherein the programmable DNA modification system is introduced to the eukaryotic cell in the presence of two or more polyanionic polymers or salts thereof.

7. The method of claim 1, wherein the introducing comprises one or more electroporation steps.

8. (canceled)

9. The method of claim 1, wherein the polyanionic polymer or salt thereof has an average molecular weight greater than about 5 kDa, greater than about 15 kDa, greater than about 25 kDa, greater than about 50 kDa, greater than about 75 kDa, greater than about 100 kDa, greater than about 150 kDa, greater than about 200 kDa, greater than about 250 kDa, greater than about 300 kDa, greater than about 350 kDa, greater than about 400 kDa, greater than about 450 kDa, or greater than about 500 kDa.

10. The method of claim 9, wherein the polyanionic polymer or salt thereof has an average molecular weight greater than about 500 kDa.

11. The method of claim 1, wherein the polyanionic polymer or salt thereof is a polysaccharide polymer, for example a sulfated polysaccharide polymer.

12. The method of claim 1, wherein
(i) the polyanionic polymer or salt thereof comprises one or more of carrageenan, cellulose, chondroitin, collagen, dextran, fucoidan, heparan, heparin, glucosamine, laminarin, pentosan, and combinations, derivatives, and/or salts thereof; or
(ii) the polyanionic polymer is a polysaccharide sulfate or a salt thereof, wherein the repeating saccharide unit includes at least one sulfate group, and comprising one or more of dextran sulfate, fucoidan sulfate, heparan sulfate, heparin sulfate, chondroitin sulfate, dermatan sulfate, and salts thereof.

13. The method of claim 12, wherein the polyanionic polymer or salt thereof comprises dextran sulfate and/or dextran sulfate sodium salt.

14. (canceled)

15. The method of claim 12, wherein the polyanionic polymer or salt thereof is dextran sulfate sodium salt.

16. The method of claim 1, wherein the programmable DNA modification system comprises a RNA-guided clustered regularly interspersed short palindromic repeats (CRISPR)/CRISPR-associated (Cas) (CRISPR-Cas) nuclease system, a CRISPR-Cas dual nickase system, a zinc finger nuclease (ZFN), a transcription activator-like effector nuclease (TALEN), a meganuclease, a fusion protein comprising a programmable DNA binding domain linked to a nuclease domain, or a fusion protein comprising a programmable DNA binding domain linked to a non-nuclease domain.

17. The method of claim 16, wherein the programmable DNA modification system comprises a Cas protein and a guide RNA.

18. The method of claim 17, wherein the guide RNA comprises a crRNA and a tracrRNA.

19. (canceled)

20. The method of claim 16, wherein the Cas protein is catalytically active, catalytically inactive, or a nickase.

21. (canceled)

22. The method of claim 1, wherein the programmable DNA modification system comprises a type I, type II (e.g., Cas9), type III, type V (e.g., Cpf1), or type VI (e.g., Cas13) Cas protein.

23. The method of claim 1, wherein the programmable DNA modification system comprises a Cas1 protein, a Cas2 protein, a Cas3 protein, a Cas4 protein, a Cas5 protein, a Cas6 protein, a Cas7 protein, a Cas8 protein, a Cas9 protein, a Cas10 protein, a Cas12 (Cpf1) protein, or a Cas13 protein.

24. The method of claim 23, wherein the programmable DNA binding system comprises Cas9 or Cas12 (Cpf1) a gRNA, and optionally a donor polynucleotide.

25. (canceled)

26. (canceled)

27. The method of claim 17, wherein the Cas protein comprises at least one amino acid mutation relative to a wild-type Cas protein.

28. (canceled)

29. The method of claim 17, wherein the Cas protein further comprises at least one nuclear localization signal, at least one cell-penetrating domain, at least one marker domain, or a combination thereof.

30. (canceled)

31. (canceled)

32. The method of claim 24, wherein the guide RNA comprises a crRNA and a tracrRNA.

33. The method of claim 24, wherein the guide RNA is a single molecule.

34. The method of claim 24, wherein the guide RNA is two molecules.

35. (canceled)

36. The method of claim 24, wherein the guide RNA comprises a crRNA and a tracrRNA, wherein the crRNA is chemically synthesized and the tracrRNA is enzymatically synthesized, or both the crRNA and the tracrRNA are enzymatically synthesized.

37. The method of claim 1, wherein the eukaryotic cell is a human cell, a non-human mammalian cell, a non-mammalian vertebrate cell, an invertebrate cell, a plant cell, or a single cell eukaryotic organism.

38. (canceled)

39. (canceled)

40. A eukaryotic cell, prepared in accordance with the method of claim 37.

41. A transfection composition comprising a programmable DNA modification system, a eukaryotic cell, and at least one polyanionic polymer or salt thereof.

42. The composition of claim 41, wherein the polyanionic polymer or salt thereof is a sulfated polysaccharide salt.

43. The composition of claim 41, wherein
(i) the polyanionic polymer or salt thereof comprises one or more of carrageenan (e.g., K-, I-, and/or L-carrageenans), cellulose (e.g., carboxymethylcellulose), chondroitin, collagen, dextran, fucoidan, heparan, heparin, glucosamine, laminarin, pentosan, and combinations, derivatives, and/or salts thereof; or
(ii) the polyanionic polymer is a polysaccharide sulfate or a salt thereof, wherein the repeating saccharide unit includes at least one sulfate group, and comprising one or more of dextran sulfate, fucoidan sulfate, heparan sulfate, heparin sulfate, chondroitin sulfate, dermatan sulfate, and salts thereof.

44. The composition of claim 41, wherein the polyanionic polymer or salt thereof comprises dextran sulfate and/or dextran sulfate sodium salt.

45. (canceled)

46. The composition of claim 44 wherein the polyanionic polymer or salt thereof is dextran sulfate sodium salt.

47. The composition of claim 41, wherein the programmable DNA modification system comprises a RNA-guided clustered regularly interspersed short palindromic repeats (CRISPR)/CRISPR-associated (Cas) (CRISPR-Cas) nuclease system, a CRISPR-Cas dual nickase system, a zinc finger nuclease (ZFN), a transcription activator-like effector nuclease (TALEN), a meganuclease, a fusion protein comprising a programmable DNA binding domain linked to a nuclease domain, or a fusion protein comprising a programmable DNA binding domain linked to a non-nuclease domain.

48. The composition of claim 41, wherein the programmable DNA modification system comprises a Cas protein and a guide RNA.

49. The composition of claim 48, wherein the guide RNA comprises a crRNA and a tracrRNA.

50. The composition of claim 41, wherein the programmable DNA modification system is a ribonucleoprotein complex comprising a Cas protein and a guide RNA, and optionally a donor polynucleotide.

51. The composition of claim 48, wherein the Cas protein is catalytically active, catalytically inactive, or a nickase.

52. The composition of claim 41, wherein the programmable DNA modification system is a base editor for modifying a base within a nucleic acid sequence.

53. (canceled)

54. The composition of claim 41, wherein the programmable DNA modification system comprises a Cas1 protein, a Cas2 protein, a Cas3 protein, a Cas4 protein, a Cas5 protein, a Cas6 protein, a Cas7 protein, a Cas8 protein, a Cas9 protein, a Cas10 protein, a Cas12 (Cpf1) protein, or a Cas13 protein.

55. The composition of claim 41, wherein the programmable DNA binding system comprises Cas9 or Cas12 (Cpf1), a gRNA, and optionally a donor polynucleotide.

56. (canceled)

57. (canceled)

58. The composition of claim 50, wherein the Cas protein comprises at least one amino acid mutation relative to a wild-type Cas protein.

59. (canceled)

60. The composition of claim 50, wherein the Cas protein further comprises at least one nuclear localization signal, at least one cell-penetrating domain, at least one marker domain, or a combination thereof.

61. (canceled)

62. (canceled)

63. The composition of claim 55, wherein the guide RNA comprises a crRNA and a tracrRNA.

64. The composition of claim 55, wherein the guide RNA is a single molecule.

65. The composition of claim 55, wherein the guide RNA is two molecules.

66. (canceled)

67. The composition of claim 55, wherein the guide RNA comprises a crRNA and a tracrRNA, wherein the crRNA is chemically synthesized and the tracrRNA is enzymatically synthesized, or both the crRNA and the tracrRNA are enzymatically synthesized.

68. The composition of claim 41, wherein the eukaryotic cell is a human cell, a non-human mammalian cell, a non-mammalian vertebrate cell, an invertebrate cell, a plant cell, or a single cell eukaryotic organism.

69. (canceled)

70. (canceled)

71. A transfection composition comprising a programmable DNA modification system, a eukaryotic cell, and two or more polyanionic polymers or salts thereof.

72. The composition of claim 71, wherein the two or more polyanionic polymers or salts thereof are sulfated polysaccharide salts.

73. The composition of claim 71, wherein
(i) the two or more polyanionic polymers or salts thereof are selected from one or more of carrageenan (e.g., K-, I-, and/or L-carrageenans), cellulose (e.g., carboxymethylcellulose), chondroitin, collagen, dextran, fucoidan, heparan, heparin, glucosamine, laminarin, pentosan, and combinations, derivatives, and/or salts thereof; or
(ii) the two or more polyanionic polymers or salts thereof are polysaccharide sulfates or salts thereof, wherein the repeating saccharide unit includes at least one sulfate group, and wherein the two or more polyanionic polymers or salts thereof are selected from dextran sulfate, fucoidan sulfate, heparan sulfate, heparin sulfate, chondroitin sulfate, dermatan sulfate, and salts thereof.

74. The composition of claim 71, wherein at least one of the two or more polyanionic polymers or salts thereof comprises dextran sulfate and/or dextran sulfate sodium salt.

75. (canceled)

76. The composition of claim 71, wherein one of the two or more polyanionic polymers or salts thereof is dextran sulfate sodium salt and another of the two or more polyanionic polymers or salts thereof is selected from fucoidan sulfate, heparan sulfate, heparin sulfate, chondroitin sulfate, and dermatan sulfate.

77. The composition of claim 71, wherein the programmable DNA modification system comprises a RNA-guided clustered regularly interspersed short palindromic repeats (CRISPR)/CRISPR-associated (Cas) (CRISPR-Cas) nuclease system, a CRISPR-Cas dual nickase system, a zinc finger nuclease (ZFN), a transcription activator-like effector nuclease (TALEN), a meganuclease, a fusion protein comprising a programmable DNA binding domain linked to a nuclease domain, or a fusion protein comprising a programmable DNA binding domain linked to a non-nuclease domain.

78. The composition of claim 71, wherein the programmable DNA modification system comprises a Cas protein and a guide RNA.

79. The composition of claim 78, wherein the guide RNA comprises a crRNA and a tracrRNA.

80. The composition of claim 71, wherein the programmable DNA modification system is a ribonucleoprotein complex comprising a Cas protein and a guide RNA, and optionally a donor polynucleotide.

81. The composition of claim 78, wherein the Cas protein is catalytically active, catalytically inactive, or a nickase.

82. The composition of claim 71, wherein the programmable DNA modification system is a base editor for modifying a base within a nucleic acid sequence.

83. (canceled)

84. The composition of claim 71, wherein the programmable DNA modification system comprises a Cas1 protein, a Cas2 protein, a Cas3 protein, a Cas4 protein, a Cas5 protein, a Cas6 protein, a Cas7 protein, a Cas8 protein, a Cas9 protein, a Cas10 protein, a Cas12 (Cpf1) protein, or a Cas13 protein.

85. The composition of claim 71, wherein the programmable DNA binding system comprises Cas9 or Cas12 (Cpf1), a gRNA, and optionally a donor polynucleotide.

86. (canceled)

87. (canceled)

88. The composition of claim 80, wherein the Cas protein comprises at least one amino acid mutation relative to a wild-type Cas protein.

89. (canceled)

90. The composition of claim 80, wherein the Cas protein further comprises at least one nuclear localization signal, at least one cell-penetrating domain, at least one marker domain, or a combination thereof.

91. (canceled)

92. (canceled)

93. The composition of claim 85, wherein the guide RNA comprises a crRNA and a tracrRNA.

94. The composition of claim 85, wherein the guide RNA is a single molecule.

95. The composition of claim 85, wherein the guide RNA is two molecules.

96. (canceled)

97. The composition of claim 85, wherein the guide RNA comprises a crRNA and a tracrRNA, wherein the crRNA is chemically synthesized and the tracrRNA is enzymatically synthesized, or both the crRNA and the tracrRNA are enzymatically synthesized.

98. The composition of claim 71, wherein the eukaryotic cell is a human cell, a non-human mammalian cell, a non-mammalian vertebrate cell, an invertebrate cell, a plant cell, or a single cell eukaryotic organism.

99. (canceled)

100. (canceled)

101. A kit comprising a Cas protein, a solution including a polyanionic polymer or salt thereof, and one or more buffer solutions.

102. The kit of claim 101, wherein the one or more buffer solutions include a reconstitution solution and a dilution solution.

103. The kit of claim 101, wherein the solution including a polyanionic polymer or salt thereof comprises dextran sulfate.

104. The kit of claim 102, wherein the reconstitution solution comprises 50% glycerol and the dilution solution comprises 20 mM HEPES buffer (pH 7.5) and 20 mM NaCl.

105. The kit of claim 103, wherein the solution including a polyanionic polymer or salt thereof comprises 1 μg/μL dextran sulfate sodium salt in 2 mM HEPES buffer (pH 7.5) and 20 mM NaCl.
